(19) United States
(12) Patent Application Publication
(10) Pub. No.: US 2021/0106629 A1
(43) Pub. Date: Apr. 15, 2021

(54) Compositions and Methods for Treating Inflammatory Bowel Diseases (IBDs) and Other Disorders

(71) Applicant: Finch Therapeutics Holdings LLC, Somerville, MA (US)

(72) Inventor: Thomas J. BORODY, Five Dock (AU)

(73) Assignee: Finch Therapeutics Holdings LLC, Somerville, MA (US)

(21) Appl. No.: 17/135,544

(22) Filed: Dec. 28, 2020

Related U.S. Application Data
(63) Continuation of application No. 15/611,338, filed on Jun. 1, 2017.
(60) Provisional application No. 62/344,053, filed on Jun. 1, 2016.

(51) Int. Cl. A61K 35/741; A61K 38/06; A61K 31/732; A61K 31/525; A61K 31/7028; A61K 31/198; A61K 31/519; A61K 9/00; A61K 35/37; A61K 35/74; A61K 47/26; A61K 47/36

(52) U.S. Cl. CPC A61K 35/741; A61K 2035/11; A61K 31/732; A61K 31/525; A61K 31/7028; A61K 31/198; A61K 31/519; A61K 9/0031; A61K 9/0053; A61K 35/37; A61K 35/74; A61K 47/26; A61K 47/36; Y02A 50/30; A61K 38/063

(57) ABSTRACT

The present disclosure is in the field of pharmaceutical compositions suitable for the treatment of diseases in mammals. This application provides novel compositions and methods for treating various disorders or conditions that are associated with a dysfunctional intestinal microbiota. In particular, this application provides compositions and methods that can treat or cure gastrointestinal (GI) disorders such as Inflammatory Bowel Disease (IBD), including, for example, Crohn's Disease and ulcerative colitis.

### CROSS-REFERENCE TO RELATED APPLICATIONS

This application claims the benefit of U.S. Provisional Application No. 62/344,053, filed Jun. 1, 2016, which is incorporated by reference in its entirety herein.

### FIELD

This application provides novel compositions and methods for treating various disorders or conditions that are associated with a dysfunctional intestinal microbiota. In particular, this application provides compositions and methods that can treat or cure gastrointestinal (GI) disorders such as Inflammatory Bowel Disease (IBD) (including, for example, Crohn's. Disease and ulcerative colitis).

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, and constipation, among other symptoms. Hence, the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

Inflammatory bowel disease (IBD) involves chronic infection and inflammation of all or part of a patient's digestive tract. IBD primarily includes ulcerative colitis and Crohn's disease. Collagenous colitis, ischemic colitis, diversion colitis, indeterminate colitis, microscopic colitis, mucous colitis, pseudomembranous colitis, and lymphocytic colitis generally are also considered inflammatory bowel diseases.

Ulcerative colitis is a chronic disease of the large intestine, also known as the colon, in which the lining of the colon becomes inflamed and develops tiny open sores, or ulcers, that produce pus and mucous. The combination of inflammation and ulceration can cause abdominal discomfort and frequent emptying of the colon. Existing treatments for ulcerative colitis involve intense and lengthy combinational drug therapy with significant side effects or even require surgery to remove part of the colon. Thus, there is a need for more effective treatments for ulcerative colitis that are easier to administer and that can cure this debilitating condition.

### SUMMARY

In one aspect, the present application provides a pharmaceutical composition comprising a plurality of live non-pathogenic microbes capable of inhibiting or antagonizing a Fusobacterium species. In another aspect, a pharmaceutical composition comprises one or more live non-pathogenic Faecalibacterium spp. In one aspect, a pharmaceutical composition comprises a fecal microbiota preparation having a donor's entire or substantially complete microbiota supplemented with one or more live non-pathogenic Faecalibacterium spp. In another aspect, a pharmaceutical composition further comprises a sterile fecal filtrate. In one aspect, a sterile fecal filtrate originates from a donor stool. In another aspect, a sterile fecal filtrate originates from cultured microorganisms.

In another aspect, the present application provides a pharmaceutical composition comprising a first plurality of live non-pathogenic microbes capable of inhibiting or antagonizing a Fusobacterium species and a second plurality of live non-pathogenic microbes capable of inhibiting or antagonizing a Mycobacterium species. In one aspect, a second plurality of live non-pathogenic microbes comprise one or more, two or more, three or more, four or more, five or more, six or more, or seven or more species selected from the anti-myco group consisting of Corynebacterium, Dietzia, Gordonia, Mycobacterium, Nocardia, Segniliparus, Skermania, Tsukamurella, Turicella, Rhodococcus, and Williamsia. In another aspect, a pharmaceutical composition comprises a fecal microbiota preparation having a donor's entire or substantially complete microbiota.

In one aspect, the present application provides a pharmaceutical composition or a kit comprising a fecal microbiota from a single donor subject administered with one or more Faecalibacterium species or one or more growth stimulating agents for at least one Faecalibacterium species, wherein the fecal microbiota comprises an elevated level of the at least one Faecalibacterium species relative to a control fecal microbiota from the same donor subject not administered with the one or more growth stimulating agents.

In another aspect, the present application provides a pharmaceutical composition comprising a non-synthetic fecal microbiota, wherein the non-synthetic fecal microbiota comprises an elevated level of at least one Faecalibacterium species relative to a control fecal microbiota from a normal healthy donor.

In one aspect, the present application provides a pharmaceutical composition comprising a non-selected fecal microbiota, wherein the non-selected fecal microbiota comprises an elevated level of at least one Faecalibacterium species relative to a control non-selected fecal microbiota from a normal healthy donor.

In another aspect, the present application provides a pharmaceutical composition comprising an untreated, non-synthetic fecal microbiota from a single donor subject, wherein the untreated, non-synthetic fecal microbiota comprises an elevated level of at least one Faecalibacterium species relative to a control fecal microbiota from a normal healthy donor.

In one aspect, the present application provides a method for treating a gastrointestinal disorder in a subject in need thereof, the method comprising administering to the subject a pharmaceutically active dose of a pharmaceutical composition described herein. In one aspect, a gastrointestinal disorder being treated is Inflammatory Bowel Disease (IBD) or Irritable Bowel Syndrome (IBS).

In another aspect, the present application provides a method for treating a disorder or condition in a subject in need thereof, the method comprising administering to the subject a pharmaceutically active dose of the pharmaceutical composition described herein and effective for treating the disorder or condition, wherein the disorder or condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-esophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.

In one aspect, the present application provides a method for treating IBD in a subject in need thereof, the method comprising administering a pharmaceutically active dose of an first antibiotic or probiotic to the subject to inhibit or antagonize a Fusobacterium species.

In another aspect, the present application provides a method for treating or curing IBD in a subject in need thereof, the method comprising: removing the subject's appendix, administering to the subject a biofilm disrupting agent, administering to the subject an antibiotic, and administering to the subject a pharmaceutically active dose of the pharmaceutical composition described herein.

In one aspect, the present application provides a method comprising: administering to a subject a growth stimulating agent for a Faecalibacterium species; and collecting a fecal sample from the subject for preparing a fecal microbiota composition.

### BRIEF DESCRIPTION OF THE DRAWINGS

FIG. 1: A phylogenetic tree showing the relationship of additional exemplary F. prausnitzii isolates to other members of Clostridium cluster IV (Ruminococcaceae) based on 16S rRNA gene sequences. Sequence accession numbers are shown in parentheses. (adapted from Lopez-Siles et al. Appl. Environ. Microbiol. 78:420-28 (2012)).

### DETAILED DESCRIPTION

Unless defined otherwise herein, terms are to be understood according to conventional usage by those of ordinary skill in the relevant art.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. As used herein, the term “substantially” as in, for example, the phrase “substantially all peptides of an array,” refers to at least 90%, preferably at least 95%, more preferably at least 99%, and most preferably at least 99.9%, of the peptides of an array. Other uses of the term “substantially” involve an analogous definition.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, the term “treating” refers to (i) completely or partially inhibiting a disease, disorder or condition, for example, by arresting its development; (ii) completely or partially relieving a disease, disorder or condition, for example, by causing regression of the disease, disorder and/or condition; or (iii) completely or partially preventing a disease, disorder or condition from occurring in a patient that may be predisposed to the disease, disorder and/or condition, but has not yet been diagnosed with it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, a “microbiota” and “flora” refer to a community of microbes that live in or on a subject's body, both sustainably and transiently, including eukaryotes, archaea, bacteria, and viruses (including bacterial viruses (i.e., phages)). A “fecal microbiota” or “fecal microbiota preparation” refers to a community of microbes present in a subject's feces. A non-selected fecal microbiota refers to a community or mixture of fecal microbes derived and processed from a donor's fecal sample without selection for any particular group or type of microbes and substantially resembling microbial constituents and population structure found in such fecal sample.

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. Preferably, a non-cellular or sterile fecal filtrate is also free of viruses for eukaryotic host cells.

As used herein, a “growth stimulating agent” or “growth stimulant” refers to a substance capable of promoting or enhancing the growth, proliferation, or viability of a target organism.

As used herein, “remission, cure, or resolution rate” refers to the percentage of patients that are cured or obtain remission or complete resolution of a condition in response to a given treatment. Remission, cure, or resolution of ulcerative colitis refers to complete cessation of rectal bleeding, urgency, and increased stool frequency. Quantitatively, remission, cure, or resolution is achieved when a patient's UCDAI score is below or equal to 2, assessed after 8 weeks of treatment. Remission, cure, or resolution can be further confirmed by endoscopic and mucosal healing.

As used herein, “response rate” refers to the percentage of patients that respond positively (e.g., reduced severity or frequency of one or more symptoms) to a given treatment. Quantitatively, a patient responds to a treatment positively when the patient's UCDAI score decreases by at least 2 from baseline to week 8.

As used herein, “ulcerative colitis disease activity index” or “UCDAI” refers to an index system for assessing the symptomatic severity or response of a ulcerative colitis patient. The index assesses four variables, which include stool frequency, severity of bleeding, colonic mucosal appearance, and the physician's overall assessment of disease activity (Table 1). See Sutherland et al., 5-Aminosalicylic acid enema in the treatment of distal ulcerative colitis, proctosigmoiditis, and proctitis. Gastroenterology. 1987; 92:1894-8. Each variable is scored from 0-3 so that the total index score ranges from 0-12; 0-2: remission; 3-6: mild; 7-10: moderate; >10: severe ulcerative colitis.

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample. The number of cfu can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium indicates the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

TABLE 1

| | score |
|---|---|
| | assignment |
| 1. Stool frequency | |
| Normal | 0 |
| 1-2 Stools/day > normal | 1 |
| 3-4 Stools/day > normal | 2 |
| >4 Stools/day > normal | 3 |
| 2. Rectal bleeding | |
| None | 0 |
| Streaks of blood | 1 |
| Obvious blood | 2 |
| Mostly blood | 3 |
| 3. Mucosal appearance | |
| Normal | 0 |
| Mild friability | 1 |
| Moderate friability | 2 |
| Exudation, spontaneous bleeding | 3 |
| 4. Physician's rating of disease activity | |
| Normal | 0 |
| Mild | 1 |
| Moderate | 2 |
| Severe | 3 |

Ulcerative colitis (UC) disease activity index. See Tursi et at., Am J Gastroentero, 105: 2218-27 (2010).

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula H=−Σᵢ₌₁^{R}ln pᵢ where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered; for example, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time; for example, once per day, once a week, or once in two weeks.

As used herein, “dosing interval” refers to the amount of time that elapses between multiple doses being administered to a subject.

Ulcerative colitis is a disease that is characterized by inflammation and micro-ulcers in the superficial layers of the large intestine. The inflammation usually occurs in the rectum and lower part of the colon, but it may affect the entire large intestine (pancolitis). Ulcerative colitis can very rarely affect the small intestine in its distal portion (Backwash Ileitis).

The inflammation is accompanied usually with diarrhoea, which may be profuse and bloody. Micro-ulcers form in places where inflammation has destroyed the cells lining the bowel; these areas bleed and produce pus and mucus. Ulcerative colitis, especially when mild, can be difficult to diagnose because symptoms are similar to other intestinal disorders, most notably the other type of IBD called Crohn's disease and also irritable bowel syndrome. Crohn's disease differs from ulcerative colitis because it causes inflammation throughout the whole thickness of the intestinal wall and produces deep ulcers. Crohn's disease usually occurs in the small intestine, but it can also occur in the large intestine, anus, oesophagus, stomach, appendix and mouth. Crohn's disease causes fistulae, whereas ulcerative colitis does not. Crohn's and ulcerative colitis may co-exist in the same patient.

Ulcerative colitis occurs most often in people ages 15 to 30, although the disease may afflict people of any age. It affects men and women equally and appears to run in some families.

Different types of ulcerative colitis exist. As used herein, “ulcerative proctitis” refers to a disease form where bowel inflammation is limited to the rectum. Because of its limited extent (usually less than the six inches of the rectum), ulcerative proctitis tends to be a milder form of ulcerative colitis. It is associated with fewer complications and offers a better outlook than more widespread disease. For approximately 30% of patients with ulcerative colitis, the illness begins as ulcerative proctitis.

As used herein, “proctosigmoiditis” refers to a form of colitis affecting the rectum and the sigmoid colon, the lower segment of colon located right above the rectum. Symptoms include bloody diarrhea, cramps, and a constant feeling of the need to pass stool, known as tenesmus. Moderate pain on the lower left side of the abdomen may occur in active disease.

As used herein, “left-sided colitis” refers to continuous inflammation that begins at the rectum and extends as far as a bend in the colon near the spleen called the splenic flexure. Symptoms include loss of appetite, weight loss, diarrhea, severe pain on the left side of the abdomen, and bleeding.

As used herein, “pan-ulcerative (total) colitis” affects the entire colon. Symptoms include diarrhea, severe abdominal pain, cramps, and extensive weight loss. Potentially serious complications include massive bleeding and acute dilation of the colon (toxic megacolon), which may lead to an opening in the bowel wall. Serious complications may require surgery.

Several theories have been proposed regarding the cause of ulcerative colitis. There is some evidence to suggest that the body's immune system reacts to an environmental, dietary or infectious agent in genetically susceptible individuals causing inflammation in the intestinal wall. Ulcerative colitis is not caused by emotional distress or sensitivity to certain foods or food products but these factors may trigger symptoms in some people. Ulcerative colitis is most likely not an aberrant reaction but an infection.

The most common symptoms of ulcerative colitis are bloody diarrhoea and abdominal pain. Patients also may experience fever, rectal bleeding, fatigue, anemia, loss of appetite, weight loss and loss of body fluids and nutrients resulting in nutritional deficiencies. These symptoms occur as intermittent attacks (flare-ups) in between periods when the symptoms go away (remissions). These disease-free periods can last for months or even years. Usually a flare-up begins with increased urgency to defecate, mild lower abdominal cramps, and blood and mucus in the stools.

Ulcerative colitis may cause long-term problems such as arthritis, inflammation of the eye, liver disease (fatty liver, hepatitis, cirrhosis, and primary sclerosing cholangitis), osteoporosis, skin rashes, anaemia and kidney stones. These complications may occur when the immune system triggers inflammation in other parts of the body. These problems can disappear when the colitis is treated effectively.

Treatment for ulcerative colitis depends on the seriousness of the disease. Most people are treated with medication. Some people whose symptoms are triggered by certain foods are able to control the symptoms by avoiding foods that upset their intestines, like highly seasoned foods or dairy products. Each person experiences ulcerative colitis differently, so treatment is adjusted for each individual.

Many patients with mild or moderate disease are first treated with 5-ASA agents, including a combination of the drugs 5-aminosalicylic acids and sulfasalazine that helps control inflammation. Sulfasalazine is the most commonly used of these drugs. Sulfasalazine can be used for as long as needed and can be given along with other drugs. Patients who do not do well on sulfasalazine may respond to newer 5-ASA agents. Possible side effects of 5-ASA preparations include nausea, vomiting, heartburn, diarrhoea and headache.

People with severe disease and those who do not respond to 5-ASA preparations may be treated with added corticosteroids. Prednisone, budesonide, and hydrocortisone are corticosteroids used to reduce inflammation. They can be given orally, intravenously, through an enema, or in a suppository, depending on the location of the inflammation. Corticosteroids can cause side effects such as weight gain, acne, facial hair, hypertension, diabetes, mood swings, and increased risk of infection, so doctors carefully monitor patients taking these medications.

Immunosuppressants such as azathioprine, 6-mercaptopurine (6-MP) and methotrexate are often used and can make a marked improvement at a low dose with few side effects. Other drugs may be given to relax the patient or to relieve pain, diarrhoea, or infection. Occasionally, symptoms are severe enough that the person must be hospitalized. For example, a person may have severe bleeding or severe diarrhoea that causes dehydration. In such cases the doctor will try to stop diarrhoea and loss of blood, fluids, and mineral salts. The patient may need a special diet, feeding through a vein, medications, or sometimes surgery.

In severe cases, a patient may need surgery to remove the diseased colon. Sometimes the doctor will recommend removing the colon if medical treatment fails or if the side effects of corticosteroids or other drugs threaten the patient's health.

Previous observations assert a potential association between certain Fusobacterium intestinal infection (e.g., by F. varium, F. nucleatum, or F. necrophorum) and ulcerative colitis. See, e.g., Ohkusa et al., Gut. 52(1): 79-83(2003); Sasaki and Klapproth, Journal of Signal Transduction, vol. 2012, Article ID 704953, 6 pages, (2012); Allen-Vercoe, Digestive Diseases and Sciences, 60(1):7-8 (2015). Fusobacteria are anaerobic gram-negative bacilli, non-sporulating, slender cells with tapered ends or pleomorphism. Fusobacterium spp. are part of the normal flora of the oropharyngeal, gastrointestinal and genital tracts. Infections may occur after surgical or accidental trauma, edema, anoxia, tissue destruction, and animal bites. In particular, F. varium was asserted to be present in the colonic mucosa of a high proportion (84%) of ulcerative colitis patients, as compared to Crohn's disease (16%) or other controls (3-13%). See Ohkusa et al. J Gastroenterol Hepatol 17:849-53 (2002). Ohkusa et al. (2003) also asserted the induction of experimental ulcerative colitis in a mouse model by F. varium isolated from colonic mucosa of ulcerative colitis patients. Gut. 52(1): 79-83 (2003).

Meanwhile, F. nucleatum has also been asserted to be associated with appendicitis. See Allen-Vercoe et al., Gut Microbes. 2:294-98 (2011). For example, a local invasion of F. nucleatum and F. necrophorum was observed in acute appendicitis. See Swidsinski et al., Gut, 60:34-40 (2011). Specifically, Swidsinski et al. studied sections of 70 appendixes with confirmed appendicitis using rRNA-based fluorescence in situ hybridization and found bacteria deeply infiltrating the appendix. Fusobacteria (mainly F. nucleatum and F. necrophorum) were specific components of epithelial and submucosal infiltrates in 62% of patients and were not found in various controls. The presence of fusobacteria correlated positively with the severity of appendicitis. Conversely, main fecal microbiota including Bacteroides, Eubacterium rectale (Clostridium group XIVa), Faecalibacterium prausnitzii groups and Akkermansia muciniphila were significantly decreased with an inverse relationship with the severity of appendicitis.

Studies have been reported analyzing the association between appendectomy and ulcerative colitis and the majority of these studies assert a highly significant inverse relationship. See Roblin et al., Gut, 61:635-36(2012). In particular, it was asserted that appendectomy for an inflammatory condition (appendicitis or lymphadenitis) but not for nonspecific abdominal pain is associated with a low risk of subsequent ulcerative colitis. Further, this inverse relation is limited to patients who undergo surgery before the age of 20. See Andersson et al., N Engl Med; 344:808-14 (2001). Therefore, the development of an appendiceal dysbiosis may be a priming event in the occurrence of ulcerative colitis. The removal of the appendix may reduce the risk of further development of ulcerative colitis in genetically susceptible individuals.

Despite the above assertion of a correlation between Fusobacterium intestinal infections and IBDs (e.g., ulcerative colitis), to the best knowledge of the applicant, it has neither been tested nor demonstrated until the present application that inhibiting a Fusobacterium intestinal infection can treat an IBD.

In one aspect, this disclosure provides a pharmaceutical composition and a method or use thereof for treating an IBD or other conditions in a subject in need thereof, where the composition comprises a plurality of live non-pathogenic microbes capable of inhibiting or antagonizing a Fusobacterium species. In one aspect, a pharmaceutical composition comprises a plurality of live non-pathogenic microbes that exhibit cytotoxic or cytostatic activity against one or more Fusobacterium species. In one aspect, a pharmaceutical composition comprises a plurality of live non-pathogenic microbes from a synthetic culture. In one aspect, a Fusobacterium species being inhibited is selected from the group consisting of F. necrophorum, F. nucleatum, F. canifelinum, F. gonidiaformans, F. mortiferum, F. naviforme, F. necrogenes, F. russii, F. ulcerans, and F. varium. In another aspect, a Fusobacterium species being inhibited is selected from the group consisting of F. nucleatum, F. necrophorum, and F. varium.

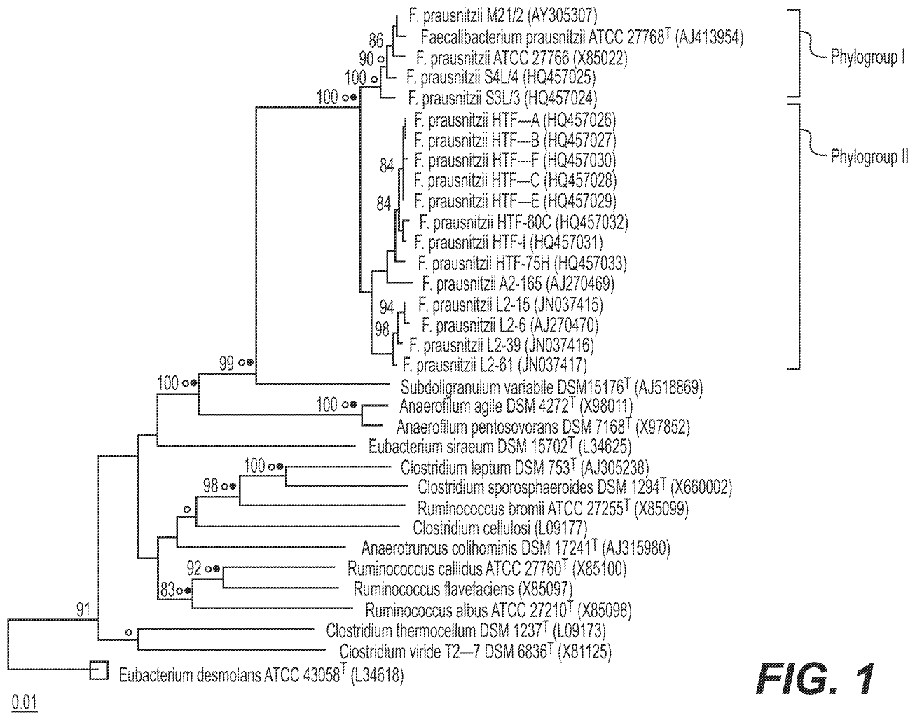

Faecalibacterium prausnitzii is one of the most abundant bacteria in the human gut ecosystem with numbers ranging from 5-20% of the total microbiota in stools of healthy individuals. F. prausnitzii is an important supplier of butyrate to the colonic epithelium. F. prausnitzii was initially classified as Fusobacterium prausnitzii. However, the complete sequence of the 16s rRNA gene of different human strains (ATCC 27766 and ATCC 27768) established that they were only distantly related to Fusobacterium and were more closely related to members of Clostridium cluster IV (the Clostridium leptum group). In 2002, Duncan et al. proposed that a new genus Faecalibacterium be created to include the non-spore-forming and non-motile Gram positive bacterium named Faecalibacterium prausnitzii. See Duncan et al., Int J Sys Evol Microbiol 52: 2141-46 (2002). According to a 16S rRNA phylogenetic analysis, the genus Faecalibacterium is a member of the family Ruminococcaceae, order Clostridiales, class Clostridia in the phylum Firmicutes. F. prausnitzii has been considered a strict anaerobe that loses its viability within two minutes after exposure to ambient air. See Duncan et al., Int J Sys Evol Microbiol 52: 2141-46 (2002). Exemplary Faecalibacterium species and strains are listed in Table 2 and FIG. 1.

TABLE 2
Exemplary Faecalibacterium.

Faecalibacterium prausnitzii
Faecalibacterium prausnitzii A2-165
Faecalibacterium prausnitzii ATCC 27768
Faecalibacterium prausnitzii ATCC 27766
Faecalibacterium cf. prausnitzii KLE1255
Faecalibacterium prausnitzii L2-6
Faecalibacterium prausnitzii M21/2
Faecalibacterium prausnitzii SL3/3
Faecalibacterium sp. canine oral taxon 147
Faecalibacterium sp. DJF_VR20
Faecalibacterium sp. MC_41
Faecalibacterium sp. CAG: 1138
Faecalibacterium sp. CAG: 74
Faecalibacterium sp. CAG: 82

F. prausnitzii was asserted to exhibit anti-inflammatory effects in vitro and in vivo using a mouse colitis model. In particular, administering F. prausnitzii strain A2-165 and its culture supernatant was claimed to protect against 2,4,6-trinitrobenzenesulfonic acid (TNBS)-induced colitis in mice. See Sokol et al., Proc Natl Acad Sci USA 105:16731-36 (2008). Further, Varela et al. asserted an association between F. prausnitzii levels and maintenance of remission in ulcerative colitis (UC). F. prausnitzii numbers were observed to be lower in UC patients in remission than in healthy controls. Meanwhile, F. prausnitzii numbers appeared higher in UC patients who remain in remission than in those who relapse. See Varela, et al., Alimentary pharmacology & therapeutics, 38:151-61 (2013). Besides F. prausnitzii, Machiels et al. also reported a decrease of another butyrate-producing species Roseburia hominis ulcerative colitis patients and asserted that Roseburia hominis might also play a role in UC. See Machiels et al. Gut, 63:1275-83 (2014).

Again, despite the foregoing observations or reports over F. prausnitzii, to the best knowledge of the applicant, it has neither been suggested nor tested, let alone demonstrated, until the present application that live F. prausnitzii can be used to treat an IBD. Without being bound by any scientific theory, live F. prausnitzii inhibits or antagonizes the growth of Fusobacterium in an intestinal infection.

In one aspect, this disclosure provides a pharmaceutical composition and a method or use thereof for treating IBD or other conditions in a subject in need thereof, where the composition comprises a plurality of live non-pathogenic microbes comprising one or more Faecalibacterium species. In one aspect, a pharmaceutical composition comprises a plurality of live non-pathogenic Faecalibacterium prausnitzii. In one aspect, a pharmaceutical composition comprises a plurality of live non-pathogenic microbes comprising one or more, two or more, three or more, four or more, five or more, six or more, or seven or more species, isolates, or strains selected from the group consisting of Faecalibacterium prausnitzii A2-165, Faecalibacterium prausnitzii ATCC 27768, Faecalibacterium prausnitzii ATCC 27766, Faecalibacterium cf. prausnitzii KLE1255, Faecalibacterium prausnitzii L2-6, Faecalibacterium prausnitzii M21/2, Faecalibacterium prausnitzii SL3/3, Faecalibacterium sp. canine oral taxon 147, Faecalibacterium sp. DJF_VR20, Faecalibacterium sp. MC_41, Faecalibacterium sp. CAG:1138, Faecalibacterium sp. CAG:74, and Faecalibacterium sp. CAG:82. In one aspect, a pharmaceutical composition comprises a plurality of live non-pathogenic microbes comprising one or more, two or more, three or more, four or more, five or more, six or more, or seven or more Faecalibacterium species, isolates, or strains listed in FIG. 1.

In another aspect, a pharmaceutical composition also comprises a growth stimulant for Faecalibacterium. In one aspect, a pharmaceutical composition comprises a growth stimulant selected from the group consisting of apple pectin, N-acetyl glucosamine, cysteine, glutathione, riboflavin, and flavin. Most F. prausnitzii strains grow well under anaerobic conditions on apple pectin. See Lopez-Siles et al. Appl. Environ. Microbiol. 78:420-28 (2012). Pectin is extensively fermented in the human colon. Some F. prausnitzii strains use uronic acids for growth. Therefore, the instant application uses pectin-rich substrates or uronic acid to further enhance a prebiotic approach for stimulating F. prausnitzii growth and therapeutic effects.

The instant application also provides the use of stool donor as bioreactors for creating modified full spectrum microbiota to serve as a more effective therapeutic. In one aspect, the instant disclosure further provides a method comprising administering to a subject a growth stimulating agent for a Faecalibacterium species; and collecting a fecal sample from said subject for preparing a fecal microbiota composition, wherein said fecal microbiota composition comprises an elevated level of said Faecalibacterium species relative to a control fecal microbiota composition from the same subject without taking said growth stimulating agent. In one aspect, a subject orally ingests said growth stimulating agent. In one aspect, a growth stimulating agent is selected from the group consisting of apple pectin, N-acetyl glucosamine, cysteine, glutathione, riboflavin, and Flavin. See US2015/0283144. In one aspect, a fecal sample is collected at least about 1, 2, 3, 4, 5, 6, 7, 8, 9, 10, 11, 12, 13, 14, 18, 20, 24, 28, 30, or 36 hours after said administrating of said growth stimulating agent. In another aspect, a fecal sample is collected at least about 1, 2, 3, 4, 5, or 6 days after said administrating of said growth stimulating agent. In one aspect, a growth stimulating agent is administered to said subject for more than about 1, 2, 3, 4, 5, 6, 7, 8, 9, 10, 11, 12, 13, or 14 days before collecting said fecal sample. In another aspect, a growth stimulating agent is administered to said subject for more than 1, 2, 3, 4, 5, 6, 7, 8, 9, 10, 11, 12, 13, or 14 weeks before collecting said fecal sample. In one aspect, a growth stimulating agent is administered to said subject at least 1, 2, 3, 4, 5, 6, 7, 8, 9, or 10 times daily. In one aspect, a fecal microbiota composition prepared by a described method comprises 1.5-fold or more, 2-fold or more, 2.5-fold or more, 3-fold or more, 3.5-fold or more, 4-fold or more, 5-fold or more, 10-fold or more, 50-fold or more, 100-fold or more, 1000-fold or more, 10,000-fold or more Faecalibacterium compared to a control fecal microbiota from the same subject without the administering of said growth stimulating agent. In another aspect, a fecal microbiota composition prepared by a described method comprises at least 10% more, 15% more, 20% more, 25% more, 30% more, 40% more, 50% more, 60% more, 70% more, 80% more, 90% more, 100% more, 150% more, 200% more, 250% more, 300% more, 350% more, 400% more, 450% more, 500% more, 600% more, 700% more, or 800% more Faecalibacterium relative to a control fecal microbiota from the same subject without the administering of said growth stimulating agent. In another aspect, the instant disclosure further provides a method comprising: administering to a subject one or more Faecalibacterium species; and collecting a fecal sample from said subject for preparing a fecal microbiota composition, wherein said fecal microbiota composition comprises an elevated level of said one or more Faecalibacterium species relative to a control fecal microbiota composition from the same subject without taking said one or more Faecalibacterium species.

In one aspect, the instant disclosure provides a pharmaceutical composition comprising a fecal microbiota from a single donor subject administered with one or more growth stimulating agents for at least one Faecalibacterium species, wherein said fecal microbiota comprises an elevated level of said at least one Faecalibacterium species relative to a control fecal microbiota from the same donor subject not administered with said one or more growth stimulating agents. In one aspect, a donor subject ingests one or more growth stimulating agents. In another aspect, one or more growth stimulating agents are selected from the group consisting of apple pectin, N-acetyl glucosamine, cysteine, glutathione, riboflavin, and Flavin. In one aspect, a fecal microbiota is from a fecal sample collected at least about 1, 2, 3, 4, 5, 6, 7, 8, 9, 10, 11, 12, 13, 14, 18, 20, 24, 28, 30, or 36 hours after the administration of said growth stimulating agent. In another aspect, a fecal microbiota is from a fecal sample collected at least about 1, 2, 3, 4, 5, or 6 days after said administrating of said growth stimulating agent. In one aspect, one or more growth stimulating agents are administered to said donor subject for more than about 1, 2, 3, 4, 5, 6, 7, 8, 9, 10, 11, 12, 13, or 14 days. In one aspect, one or more growth stimulating agents are administered to said donor subject for more than about 1, 2, 3, 4, 5, 6, 7, 8, 9, 10, 11, 12, 13, or 14 weeks. In one aspect, one or more growth stimulating agents are administered to said donor subject at least 1, 2, 3, 4, 5, 6, 7, 8, 9, or 10 times daily. In another aspect, the instant disclosure provides a treatment method for treating an IBD (e.g., UC) in a patient in need thereof, where the method comprises administering one or more Faecalibacterium growth stimulating agents, one or more bile acid sequestrant (e.g., cholestyramine, colestipol, colesevelam), or both, to the patient, and further administering a pharmaceutical composition described herein (e.g., a non-selected fecal microbiota from a single donor subject administered with one or more Faecalibacterium growth stimulating agents).

In one aspect, the instant disclosure provides a pharmaceutical composition comprising a non-synthetic fecal microbiota, wherein said non-synthetic fecal microbiota comprises an elevated level of at least one Faecalibacterium species relative to a control fecal microbiota from a normal healthy donor. In another aspect, the instant disclosure provides a pharmaceutical composition comprising a non-selected fecal microbiota, wherein said non-selected fecal microbiota comprises an elevated level of at least one Faecalibacterium species relative to a control non-selected fecal microbiota from a normal healthy donor. In another aspect, the instant disclosure provides a pharmaceutical composition comprising an untreated, non-synthetic fecal microbiota from a single donor subject, wherein said untreated, non-synthetic fecal microbiota comprises an elevated level of at least one Faecalibacterium species relative to a control fecal microbiota from a normal healthy donor.

In one aspect, an elevated level described herein is selected from the group consisting of 1.5-fold or more, 2-fold or more, 2.5-fold or more, 3-fold or more, 3.5-fold or more, 4-fold or more, 5-fold or more, 10-fold or more, 50-fold or more, 100-fold or more, 1000-fold or more, and 10,000-fold or more. In another aspect, an elevated level described herein is selected from the group consisting of at least 10% more, at least 15% more, at least 20% more, at least 25% more, at least 30% more, at least 40% more, at least 50% more, at least 60% more, at least 70% more, at least 80% more, at least 90% more, at least 100% more, at least 150% more, at least 200% more, at least 250% more, at least 300% more, at least 350% more, at least 400% more, at least 450% more, at least 500% more, at least 600% more, at least 700% more, and at least 800% more.

Mycobacterium avium, subspecies paratuberculosis (MAP) causes a chronic disease of the intestines in dairy cows and a wide range of other animals, including nonhuman primates, called Johne's (“YO-knee's”) disease. MAP has been consistently identified by a variety of techniques in humans with Crohn's disease. The research investigating the presence of MAP in patients with Crohn's disease has often identified MAP in the “negative” ulcerative colitis controls as well, suggesting that ulcerative colitis is also caused by MAP. For example, one study reported that when multiple specimens were obtained from different sites in the intestine, the prevalence of Mycobacterium avium subspecies paratuberculosis infection was 82.1% and 40% respectively in Crohn's disease and ulcerative colitis patients. See Pistone et al., Digestive and Liver Disease 44:461-65 (2012). Besides this observation, Pierce previously hypothesized that MAP may be an etiologic agent of both ulcerative colitis and Crohn's disease, in an attempt to explain both diseases' common epidemiology, geographic distribution and familial and sporadic clusters. See Pierce, Gut Pathog. 2: 21 (2010). According to Pierce, when infected with MAP, individuals develop ulcerative colitis or Crohn's disease depending on the dose, route, age, sex and genes on the clinical expression of a MAP infection.

Dietzia subspecies C79793-74, previously known as Mycobacterium gordonae, was reported to inhibit MAP under in vitro culture conditions. See Richards (1989) in Johne's Disease: Current trends in research, diagnosis and management, Proceedings of a conference held at the Veterinary Research Institute, Parkville, Victoria. Australia, edited by Milner and Wood; Click and Van Kampen, J. Dairy Sci. 92:4846-51 (2009); U.S. Pat. No. 8,414,886. Together with Mycobacterium, Dietzia belongs to a group of mycolic acid-containing actinomycetes including genera such as Corynebacterium, Dietzia, Gordonia, Mycobacterium, Nocardia, Segniliparus, Skermania, Tsukamurella, Turicella, Rhodococcus, and Williamsia (collectively, “anti-myco bacteria” hereinafter). See Bergey's Manual of Systematic Bacteriology, Volume 5 (2012): The Actinobacteria, at pages 246, 291, 302, 313, 378, 436, 438, 470, and 501.

Combining the above explained links between Fusobacterium, appendicitis, appendectomy, ulcerative colitis, MAP, anti-myco bacteria, and Crohn's Disease, the instant application further provides the following compositions and methods for treating and curing Crohn's Disease and ulcerative colitis.

In one aspect, this disclosure provides a pharmaceutical composition and a method or use thereof for treating IBD or other conditions in a subject in need thereof, where the composition comprises a first plurality of live non-pathogenic microbes capable of inhibiting or antagonizing a Fusobacterium species and a second plurality of live non-pathogenic microbes capable of inhibiting or antagonizing a Mycobacterium species. In one aspect, a first plurality of live non-pathogenic microbes that exhibit cytotoxic or cytostatic activity against one or more Fusobacterium species. In one aspect, the first, second, or both plurality of live non-pathogenic microbes are from a synthetic culture. In one aspect, a Fusobacterium species being inhibited is selected from the group consisting of F. necrophorum, F. nucleatum, F. canifelinum, F. gonidiaformans, F. mortiferum, F. naviforme, F. necrogenes, F. russii, F. ulcerans, and F. varium. In another aspect, a Fusobacterium species being inhibited is selected from the group consisting of F. nucleatum, F. necrophorum, and F. varium. In one aspect, a Mycobacterium species being inhibited is Mycobacterium avium ssp. paratuberculosis (MAP). In one aspect, a first plurality of live non-pathogenic microbes, a second plurality of live non-pathogenic microbes, or both combined, are in an anaerobic package or container. In another aspect, a first plurality of live non-pathogenic microbes, a second plurality of live non-pathogenic microbes, or both combined, are in an aerobic package or container.

In one aspect, a first plurality of live non-pathogenic microbes comprise one or more Faecalibacterium species. In one aspect, a first plurality of live non-pathogenic microbes comprise Faecalibacterium prausnitzii. In one aspect, a first plurality of live non-pathogenic microbes comprise one or more, two or more, three or more, four or more, five or more, six or more, or seven or more species, isolates, or strains selected from the group consisting of Faecalibacterium prausnitzii A2-165, Faecalibacterium prausnitzii ATCC 27768, Faecalibacterium prausnitzii ATCC 27766, Faecalibacterium cf. prausnitzii KLE1255, Faecalibacterium prausnitzii L2-6, Faecalibacterium prausnitzii M21/2, Faecalibacterium prausnitzii SL3/3, Faecalibacterium sp. canine oral taxon 147, Faecalibacterium sp. DJF_VR20, Faecalibacterium sp. MC_41, Faecalibacterium sp. CAG:1138, Faecalibacterium sp. CAG:74, and Faecalibacterium sp. CAG:82. In one aspect, a first plurality of live non-pathogenic microbes comprise one or more, two or more, three or more, four or more, five or more, six or more, or seven or more Faecalibacterium species, isolates, or strains listed in FIG. 1. In another aspect, a pharmaceutical composition also comprises a growth stimulant for Faecalibacterium. In one aspect, a pharmaceutical composition comprises a growth stimulant selected from the group consisting of apple pectin, N-acetyl glucosamine, cysteine, glutathione, riboflavin, and flavin. In one aspect, a pharmaceutical composition is in an anaerobic package or container. In another aspect, a pharmaceutical composition further comprises an oxygen scavenger.

In one aspect, a second plurality of live non-pathogenic microbes exhibit cytotoxic or cytostatic activity against one or more Mycobacterium species. In one aspect, the cytotoxic or cytostatic activity is against a Mycobacterium species selected from the group consisting of M. tuberculosis, M. leprae, M. avium-intracellulare, M. bovis, M. cheloiei, M. africanum, M. marinium, M. buruli, M. fortuitum, M. haemophilum, M. intracellulare, M. kansasii, M. littorals, M. malmoense, M. marianum, M. sinuae, M. szulgai, and M. ulcerans, M. avium, M. flavascens, M. lepraemurium, or M. nicroti. In one aspect, the cytotoxic or cytostatic activity is against MAP.

In one aspect, a second plurality of live non-pathogenic microbes comprise one or more, two or more, three or more, four or more, five or more, six or more, or seven or more species selected from the group consisting of Corynebacterium, Dietzia, Gordonia, Mycobacterium, Nocardia, Segnihparus, Skermania, Tsukamurella, Turicella, Rhodococcus, and Williamsia (collectively, “anti-Myco” group). In another aspect, a second plurality of live non-pathogenic microbes comprise one or more, two or more, three or more, or four or more species selected from the group consisting of Dietzia, Gordonia, Mycobacterium, Nocardia, and Rhodococcus. Bacteria of the ‘anti-Myco’ group are aerobic. In one aspect, these bacteria are co-administered with growth-promoting agents such as trehalose, mannose, fructose, or D-glucose to better survive the largely anaerobic gut environment. In another aspect, iron, copper, and zinc components are added to enhance growth of these bacteria, as can the use of added moderately aged coconut (Cocos nucifera L.) water. Coconut water, originates in the coconut fruit after one and half months, is a highly nourishing liquid, containing an enormous range of chemical components: different sugars, sugar alcohols, inorganic ions, vitamins, lipids, amino acids, nitrogenous compounds, organic acids, enzymes, phytohormones, etc. Coconut water promotes the growth of anti-Myco bacteria.

In another aspect, a second plurality of live non-pathogenic microbes comprise one or more, two or more, three or more, four or more, five or more, six or more, or seven or more Dietzia species, strains, or isolates. In another aspect, a second plurality of live non-pathogenic microbes comprise one or more, two or more, three or more, four or more, five or more, six or more, or seven or more species selected from the group consisting of D. aerolata, D. alimentaria, D. aurantiaca, D. cerdiciphylli, D. cinnamea, D. kunjamensis, D. lutea, D. maris, D. natronolimnaea, D. papillomatosis, D. psychralcahphila, D. schimae, and D. timorensis.

In one aspect, a second plurality of live non-pathogenic microbes comprise one or more, two or more, three or more, four or more, five or more, six or more, or seven or more Gordonia species selected from the group consisting of G. aichiensis, G. alkanivorans, G. amarae, G. amicalis, G. araii, G. bronchialis, G. defluvii, G. desulfuricans, G. effusa, G. hirstuta, G. hydrophobica, G. lacunae, G. malaquae, G. namibiensis, G. otitidis, G. paraffinivorans, G. polyisoprenivorans, G. rhizosphera, G. rubripertincta, G. shandongensis, G. sihwensis, G. sinesedis, G. soli, G. sputi, G. terrae, and G. westfalica.

In one aspect, a second plurality of live non-pathogenic microbes comprise one or more, two or more, three or more, four or more, five or more, six or more, or seven or more Corynebacterium species selected from the group consisting of C. accolens, C. afermentans ssp. afermentans, C. ammoniagenes, C. amycolatum, C. appendicis, C. aquilae, C. argentoratense, C. atypicum, C. aurimucosum, C. auris, C. auriscanis, C. bovis, C. callunae, C. camporealensis, C. canis, C. capitovis, C. casei, C. caspium, C. ciconiae, C. confusum, C. coyleae, C. cystitidis, C. diphtheria, C. doosanense, C. durum, C. efficiens, C. falsenii, C. felinum, C. flavescens, C. freiburgense, C. freneyi, C. glaucum, C. glucuronolyticum, C. glutamicum, C. halotolerans, C. hansenii, C. imitans, C. jeikeium, C. kroppenstedtii, C. kutscheri, C. lipophiloflavum, C. lubricantis, C. macginleyi, C. marinum, C. maris, C. massiliense, C. mastitidis, C. matruchotii, C. minutissimum, C. mucifaciens, C. mustelae, C. mycetoides, C. phocae, C. pilbarense, C. pilosum, C. propinquum, C. pseudodiphtheriticum, C. pseudotuberculosis, C. pyruviciproducens, C. renale, C. resistens, C. riegelii, C. simulans, C. singular, C. sphenisci, C. spheniscorum, C. sputi, C. stationis, C. striatum, C. suicordis, C. sundsvallense, C. terpenotabidum, C. testudinoris, C. thomssenii, C. timonense, C. tuberculostearicum, C. tuscaniense, C. ulcerans, C. ukeribovis, C. urealyticum, C. ureicelerivorans, C. variabile, C. vitaeruminis, and C. xerosis.

In one aspect, a second plurality of live non-pathogenic microbes comprise one or more, two or more, three or more, four or more, five or more, six or more, or seven or more Mycobacterium species selected from the group consisting of M. tuberculosis, M. leprae, M. avium-intracellulare, M. bovis, M. cheloiei, M. africanum, M. marinium, M. buruli, M. fortuitum, M. haemophilum, M. intracellulare, M. kansasii, M. littorale, M. malmoense, M. marianum, M. sinuae, M. szulgai, and M. ulcerans, M. avium, M. flavascens, M. lepraemurium, and M. nicroti.

In one aspect, a second plurality of live non-pathogenic microbes comprise one or more, two or more, three or more, four or more, five or more, six or more, or seven or more Nocardia species selected from the group consisting of N. abscessus, N. acidivorans, N. africana, N. alba, N. altamirensis, N. amamiensis, N. anaemiae, N. aobensis, N. araoensis, N. arthritidis, N. asiatica, N. asteroides, N. beijingensis, N. blacklockiae, N. brasiliensis, N. brevicatena, N. caishijiensis, N. calitrisensis, N. carnea, N. cerradoensis, N. concava, N. coubleae, N. crassostreae, N. cummidelens, N. cyriacigeorgica, N. elegans, N. exalbida, N. farcinica, N. flavorosea, N. fluminea, N. gamkensis, N. harensis, N. higoensis, N. ignorata, N. inohanensis, N. iowensis, N. jejuensis, N. jiangxiensis, N. jinanensis, N. kruczakiae, N. lijiangensis, N. mexicana, N. miyunensis, N. neocaledoniensis, N. niigataensis, N. ninae, N. nova, N. otitidiscaviarum, N. paucivorans, N. pigrifrangens, N. pneumoniae, N. polyresistens, N. pseudobrasiliensis, N. pseudovaccinii, N. puris, N. salmonicida, N. seriolae, N. shimofusensis, N. sienata, N. soli, N. speluncae, N. takedensis, N. tenerifensis, N. terpenica, N. testacea, N. thailandica, N. transvalensis, N. uniformis, N. vaccinii, N. vermiculata, N. veterana, N. vinacea, N. wallacei, N. xishanensis, and N. yamanashiensis.

In one aspect, a second plurality of live non-pathogenic microbes comprise one or more, two or more, three or more, four or more, five or more, six or more, or seven or more Rhodococcus species selected from the group consisting of R. aurantiacus, R. aetherivorans, R. baikonurensis, R. coprophilus, R. corynebacterioides, R. equi, R. erythropolis, R. fascians, R. globerulus, R. gordoniae, R. imtechensis, R. jostii, R. koreensis, R. kroppenstedtii, R. kunmingensis, R. kyotonensis, R. maanshanensis, R. marinonascens, R. opacus, R. percolatus, R. phenolicus, R. pyridinivorans, R. qingshengii, R. rhodnii, R. rhodochrous, R. ruber, R. triatomae, R. tukisamuensis, R. wratislaviensis, R. yunnanensis, and R. zopfii.

In one aspect, a second plurality of live non-pathogenic microbes comprise one or more, two or more, three or more, four or more, five or more, six or more, or all seven species selected from the group consisting of Skermania piniformis, Williamsia deligens, Williamsia serinedens, Williamsia maris, Williamsia marianensis, Williamsia muralis, and Williamsia faeni.

In one aspect, a second plurality of live non-pathogenic microbes comprise one or more, two or more, three or more, four or more, five or more, six or more, or seven or more Tsukamurella species selected from the group consisting of T. paurometabola, T. spumae, T. inchonensis, T. sunchonensis, T. pseudospumae, T. spongiae, T. pulmonis, T. tyrosinosolvens, and T. strandjordii.

Many chronic diseases and disorders of the GI tract have chronic infection/infestation as their underlying pathological conditions (e.g., ulcerative colitis). In one aspect, the present disclosure includes and relates to the use of a fecal microbiota, one or more microbial species therefrom, an active fragment or component therefrom for the treatment and/or prophylaxis of various disease states (e.g., ulcerative colitis) related to the presence of ‘abnormal’ microflora in the GI tract. An active fragment of a bacterium can be any active molecule isolated from such bacteria by any known method for preparing/identifying active fragments of bacteria and proteins secreted from bacteria. Such methods include but are not limited to: sonication, osmotic shock, detergent lysis, high pressure, and the transfer of appropriate DNA to other organisms, such as bacteria, plant or animal that are then used as a feed additive, as described previously. In one aspect, an active fragment or component of a bacterium is selected from the group consisting of a mycolate or a derivative thereof, a polysaccharide, a lipoglycan, a small peptide, a thiopeptide, a protein, a nucleic acid molecule, a metabolite, a cell wall component, or any combination thereof. In one aspect, an active fragment is a protein or a secretion. In another aspect, an active fragment is a secreted protein.

In one aspect, this disclosure also provides a pharmaceutical composition and a method or use thereof for treating IBD or other conditions in a subject in need thereof, where the composition comprises a fecal microbiota preparation mixed, supplemented, or enhanced with a first plurality of live non-pathogenic microbes capable of inhibiting or antagonizing a Fusobacterium species and a second plurality of live non-pathogenic microbes capable of inhibiting or antagonizing a Mycobacterium species. In one aspect, a fecal microbiota preparation comprises a donor's entire or substantially complete microbiota. In one aspect, a fecal microbiota preparation comprises a non-selected fecal microbiota. In another aspect, a fecal microbiota preparation comprises an isolated or purified population of live non-pathogenic fecal bacteria. In one aspect, the preparation of a fecal microbiota preparation involves a treatment selected from the group consisting of ethanol treatment, detergent treatment, heat treatment, irradiation, sonication, and a combination thereof. In one aspect, the preparation of a fecal microbiota preparation involves no treatment selected from the group consisting of ethanol treatment, detergent treatment, heat treatment, irradiation, and sonication. In one aspect, the preparation of a fecal microbiota preparation involves a separation step selected from the group consisting of filtering, sieving, density gradients, filtration, chromatography, and a combination thereof. In one aspect, the preparation of a fecal microbiota preparation does not require one or more separation steps selected from the group consisting of filtering, sieving, density gradients, filtration, and chromatography. In one aspect, a fecal microbiota preparation is substantially free of non-living matter. In one aspect, a fecal microbiota preparation is substantially free of acellular material selected from the group consisting of residual fiber, DNA, viral coat material, and non-viable material. In one aspect, a fecal microbiota preparation is substantially free of eukaryotic cells from said fecal microbiota's donor.

In one aspect, the present disclosure provides a method for treating a disorder (e.g., ulcerative colitis or Crohn's disease) in a subject in need thereof, where the method comprises administering to the subject a pharmaceutically active dose of a therapeutic composition comprising live non-pathogenic fecal bacteria. In another aspect, this disclosure provides for the use of a composition comprising live non-pathogenic fecal bacteria in the manufacture of a medication for the treatment of a disorder (e.g., ulcerative colitis or Crohn's disease).

In one aspect, a method is for treating a form of ulcerative colitis selected from the group consisting of ulcerative proctitis, proctosigmoiditis, left-sided colitis, and pan-ulcerative colitis.

In one aspect, a therapeutic composition comprises an isolated or purified population of live non-pathogenic fecal bacteria. In one aspect, a therapeutic composition comprises a non-selected fecal microbiota. In another aspect, a therapeutic composition comprises a non-selected and substantially complete fecal microbiota. In another aspect, a therapeutic composition comprises a full-spectrum fecal microbiota. In one aspect, a method further comprises administering a 5-aminosalicylic acid agent, a corticosteroid, an immunosuppressant, or a combination thereof. In another aspect, a method further comprises administering 5-aminosalicylic acid or a derivative thereof, sulfasalazine or a derivative thereof, or a combination thereof.

In one aspect, the present disclosure provides a method which eliminates or reduces one or more ulcerative colitis symptoms selected from the group consisting of diarrhoea, cramp, tenesmus, weight loss, bleeding, loss of appetite, abdominal pain, fever, fatigue, anaemia, inflammation, and micro-ulcers.

In one aspect, the present disclosure provides a method for treating a disorder (e.g., ulcerative colitis or Crohn's disease) in a subject in need thereof, where the method comprises administering to the subject a pharmaceutically active dose of a therapeutic composition comprising live non-pathogenic bacteria. In one aspect, the present disclosure provides a method for treating a disorder (e.g., ulcerative colitis or Crohn's disease) in a subject in need thereof, where the method comprises administering daily to the subject a pharmaceutically active dose of a therapeutic composition comprising live non-pathogenic fecal bacteria. In one aspect, a therapeutic composition is administered to a patient in need thereof at least once daily for at least two consecutive days. In one aspect, a therapeutic composition is administered at least once daily for at least 3, 4, 5, 6, 7, 8, 9, 10, 11, 12, 13, 14, or 15 consecutive days. In another aspect, a therapeutic composition is administered at least once daily for at least 1, 2, 3, 4, 5, 6, 7, 8, 9, 10, 11, or 12 consecutive weeks. In one aspect, a therapeutic composition is administered at least once daily for at most 4, 5, 6, 7, 8, 9, 10, 11, 12, 13, 14, 15, 16, 17, 18, 19, or 20 consecutive days or weeks. In another aspect, a therapeutic composition is administered at least once daily for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or for an indefinite period of time.

In one aspect, a therapeutic composition is administered to a patient in need thereof at least twice daily for at least two consecutive days. In one aspect, a therapeutic composition is administered at least twice daily for at least 3, 4, 5, 6, 7, 8, 9, 10, 11, 12, 13, 14, or 15 consecutive days. In another aspect, a therapeutic composition is administered at least twice daily for at least 1, 2, 3, 4, 5, 6, 7, 8, 9, 10, 11, or 12 consecutive weeks. In one aspect, a therapeutic composition is administered at least twice daily for at most 4, 5, 6, 7, 8, 9, 10, 11, 12, 13, 14, 15, 16, 17, 18, 19, or 20 consecutive days or week. In another aspect, a therapeutic composition is administered at least twice daily for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or for an indefinite period of time.

In one aspect, a therapeutic composition is administered to a patient in need thereof at least three times daily for at least two consecutive days. In one aspect, a therapeutic composition is administered at least three times daily for at least 3, 4, 5, 6, 7, 8, 9, 10, 11, 12, 13, 14, or 15 consecutive days. In another aspect, a therapeutic composition is administered at least three times daily for at least 1, 2, 3, 4, 5, 6, 7, 8, 9, 10, 11, or 12 consecutive weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or for an indefinite period of time.

In one aspect, the present disclosure provides a method for treating a disorder (e.g., ulcerative colitis or Crohn's disease) in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks. In another aspect, a dose described here is administered at a dosing schedule of at least once or twice daily or at least once or twice weekly for at least 3, 8, 10, or 20 weeks. In a further aspect, a dose is administered at a dosing schedule of at least once or twice daily or at least one or twice weekly for at least 4, 5, 6, 7, 11, 12, 13, 14, 15, 16, 17, 18, or 19 weeks. In one aspect, the foregoing weeks are consecutive weeks. In another aspect, the foregoing weeks are nonconsecutive weeks.

In one aspect, the present disclosure provides a method for treating a disorder (e.g., ulcerative colitis or Crohn's disease) in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or for an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every 2 days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each week. “Thrice-a-week” means that a dose is administered three times in a week, preferably on the same three days of each week.

In one aspect, a subject being treated is a subject already with a disorder (e.g., ulcerative colitis or Crohn's disease). Administration of a disclosed therapeutic composition to an asymptomatic human subject who is genetically predisposed or prone to a disorder (e.g., ulcerative colitis or Crohn's disease) is also useful in preventing the onset of clinical symptoms. A human subject genetically predisposed or prone to ulcerative colitis can be a human subject having a close family member or relative exhibiting or having suffered a disorder (e.g., ulcerative colitis or Crohn's disease). In another aspect, a subject being treated is a subject in which ulcerative colitis is to be prevented. In another aspect, a subject being treated is predisposed or susceptible to a disorder (e.g., ulcerative colitis or Crohn's disease). In another aspect, a subject being treated is a subject diagnosed as having a disorder (e.g., ulcerative colitis or Crohn's disease). In one aspect, a subject being treated is a patient in need thereof. In another aspect, a patient being treated is immunocompromised.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a method comprises administering a therapeutic composition orally, by enema, or via rectal suppository. In one aspect, a therapeutic composition administered herein is formulated as an enteric coated capsule or microcapsule, acid-resistant capsule or microcapsule, or formulated as part of or administered together with a food, a food additive, a dairy-based product, a soy-based product or a derivative thereof, a jelly, flavored liquid, ice block, ice cream, or a yogurt. In another aspect, a therapeutic composition administered herein is formulated as an acid-resistant enteric coated capsule. In another aspect, a therapeutic composition administered herein is formulated as a double-encapsulated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, foam-dried, spray-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material.

In one aspect, a therapeutic composition also comprises, or is supplemented with, a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a therapeutic bacterial or microbiota composition. In another aspect, an antibiotic comprises an antibiotic combination regimen consisting of amoxicillin, tetracycline, and metronidazole (ATM). See, e.g., Kato et al., Aliment Pharmacol Ther 39: 949-56 (2014); Koido et al., PLOS One, 9(1):e86702 (2014); Nitzan et al., World J Gastroenterol. 22(3): 1078-87 (2016).

In one aspect, a method achieves a remission, cure, response, or resolution rate of ulcerative colitis of at least about 10%, 15%, 20%, 25%, 30%, 35%, 40%, 45%, 50%, 55%, 60%, 65%, 70%, 75%, 80%, 85%, 90%, 95%, 97%, or 99%. In one aspect, a treatment method achieves a reduction of ulcerative colitis disease activity index (UCDAI) after 8 weeks of treatment by more than 2, 3, 4, 5, 6, 7, 8, 9, 10, or 11. In another aspect, a treatment method achieves a reduction of ulcerative colitis disease activity index (UCDAI) after 8 weeks of treatment by more than 2, 3, 4, 5, 6, 7, 8, 9, 10, or 11 in at least 10%, 20%, 30%, 50%, 60%, 70%, 80%, or 90% patients in a patient population. In one aspect, a treatment method achieves at least 10%, 20%, 30%, 50%, 60%, 70%, 80%, or 90% reduction of ulcerative colitis disease activity index (UCDAI) after 8 weeks of treatment compared to baseline (e.g., immediately prior to treatment). In one aspect, a treatment method achieves at least 10%, 20%, 30%, 50%, 60%, 70%, 80%, or 90% reduction of ulcerative colitis disease activity index (UCDAI) in at least 10%, 20%, 30%, 50%, 60%, 70%, 80%, or 90% patients after 8 weeks of treatment compared to baseline (e.g., immediately prior to treatment).

In a further aspect, a patient is assessed using the Disease Activity Index (DAI) or Mayo score system as described in Schroeder et al., Coated oral 5-aminosalcylic acid therapy for mildly to moderately active ulcerative colitis. N Eng J Med. 1987; 317:1625-1629. In one aspect, a treatment method achieves at least 10%, 20%, 30%, 50%, 60%, 70%, 80%, or 90% reduction of said Mayo score after 8 weeks of treatment compared to baseline (e.g., immediately prior to treatment). In one aspect, a treatment method achieves at least 10%, 20%, 30%, 50%, 60%, 70%, 80%, or 90% reduction of said Mayo score in at least 10%, 20%, 30%, 50%, 60%, 70%, 80%, or 90% patients after 8 weeks of treatment compared to baseline (e.g., immediately prior to treatment).

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cells or spores. In another aspect, a pharmaceutically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a fecal microbiota preparation described herein comprises a purified or reconstituted fecal bacterial mixture. In one aspect, a fecal microbiota preparation comprises one or more, two or more, three or more, four or more, or five or more live fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation comprises one or more, two or more, three or more, four or more, or five or more live fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In one aspect, a fecal microbiota preparation lacks or is substantially devoid of one or more, two or more, three or more, four or more, or five or more live fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation lacks or is substantially devoid of one or more, two or more, three or more, four or more, or five or live more fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a non-pathogenic (or pathogenically-attenuated) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odorless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes such as prokaryotic cells, for instance bacteria and archaea (e.g., living prokaryotic cells and spores that can sporulate to become living prokaryotic cells); eukaryotic cells such as protozoa and fungi; and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed (e.g., by undergoing microfiltration) before, after, or before and after, sieving. In another aspect, a highly purified fecal microbiota product is ultra-filtrated to remove large molecules but retain the therapeutic microflora, e.g., bacteria.

In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, execreted or otherwise liquid components of a microbiota, e.g., biologically active molecules (BAMs), which can be antibiotics or anti-inflammatories, are preserved, retained or reconstituted in a flora extract. In one aspect, a BAM is a small RNA molecule, e.g., small interfering RNAs (siRNAs), short hairpin RNAs (shRNAs), trans-acting siRNAs (ta-siRNAs), or micro RNAs (miRNAs). In another aspect, a BAM is “non-coding RNA molecule” which is an RNA molecule that does not encode a protein. Non-limiting examples of a non-coding RNA molecule include a microRNA (miRNA), a miRNA precursor, a small interfering RNA (siRNA), a siRNA precursor, a small RNA (18-26 nt in length) and precursors encoding the same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single-guide RNA (sgRNA).

In one aspect, an exemplary therapeutic composition comprises starting material from a donor from a defined donor pool, where this donor contributes a stool that is centrifuged, then filtered with very high-level filtration using either metal sieving or Millipore filters, or equivalent, to ultimately permit only cells of bacterial origin to remain, e.g., often cells less than about 5 micrometers in diameter. After the initial centrifugation, the solid material is separated from the liquid, and the solid is then filtered in progressively reducing size filters and tangential filters (e.g., using a Millipore filtration), and optionally, also comprising use of nano-membrane filtering. The filtering can also be done by sieves as described in WO 2012/122478, but also using sieves that are smaller than 0.0120 mm, down to about 0.0110 mm, which ultimately result in having only bacterial cells present.

The supernatant separated during centrifugation is now taken and filtered progressively in a filtering (e.g., a Millipore filtering or equivalent systems) to end up with liquid which is finely filtered through an about 0.22 micron filter. This removes all particulate matter, including all living matter, such as bacteria and viruses. The product is then sterile, but the aim is to remove the bacteria while keeping their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, and cinnamycin), lacticins, and other antimicrobial or anti-inflammatory compounds. In one aspect, a therapeutic composition used herein comprises a cell-free fecal filtrate enriched for bacteriophage. In one aspect, lytic bacteriophage is enriched. In another aspect, temperate bacteriophage is enriched. In one aspect, a bacteriophage is from Caudovirales. In another aspect, a bacteriophage is from Ligamenvirales. In one aspect, a bacteriophage is from a family selected from the group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttaviridae, Inoviridae, Leviviridae, Microviridae, Plasmaviridae, and Tectiviridae.

In one aspect, a therapeutic composition used here comprises a combination of bacteriophage from one or more, 2 or more, 3 or more, 4 or more, 5 or more, 6 or more, or 7 or more families selected from the group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttaviridae, Inoviridae, Leviviridae, Microviridae, Plasmaviridae, and Tectiviridae.

In one aspect, a therapeutic composition used here comprises a reconstituted fecal flora consisting essentially of a combination of a purified fecal microbiota and a non-cellular fecal filtrate. In another aspect, a therapeutic composition used here comprises a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted, and/or artificial liquid component (e.g., fecal filtrate) of the flora (the microbiota) which comprises, among others ingredients, bacterial secretory products such as bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid, lanthionine or methyllanthionine, and unsaturated amino acids dehydroalanine and 2-aminoisobutyric acid, which include thuricin (secreted by bacilli in donor stools), nisin, subtilin, epidermin, mutacin, mersacidin, actagardine, cinnamycin), a lacticin (a family of pore-forming peptidic toxins), and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pre-treatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal microbiota in a therapeutic composition comprises a donor's substantially entire or non-selected fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, and non-viable material, and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces do not include an antibiotic resistant population. In another aspect, a fecal microbiota in a therapeutic composition is derived from a synthetic fecal composition. In an aspect, a synthetic fecal composition comprises a preparation of viable flora which, preferably in proportional content, resembles normal healthy human fecal flora and does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; an acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of a disorder (e.g., IBD such as ulcerative colitis or Crohn's disease). The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be the liquid (e.g., saline), gel or solid form of diluents, adjuvant, excipients or an acid-resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, the like, and combinations thereof. In another aspect, a therapeutic composition may contain auxiliary substances such as wetting or emulsifying agents or stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of the active ingredient. In an aspect, a therapeutic composition contains about 2%-70%, 5%-60%, 10%-50%, 15%-40%, 20%-30%, 25%-60%, 30%-60%, or 35%-60% of the active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like, to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch, and mannitol. Starch and alginic acid are also good disintegrators. Surface-active agents such as sodium lauryl sulfate and dioctyl sodium sulphosuccinate are also sometimes used. Commonly used absorbents include starch and lactose. Magnesium carbonate is also useful for oily substances. For use as a binder there are, for example, gelatin, gums, starch, dextrin, polyvinyl pyrrolidone, and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In an aspect, a therapeutic composition is formulated as a delayed or gradual enteric release form. In an aspect, a delayed or gradual enteric release formulation comprises the use of cellulose acetate, polyethylene glycerol, or both. In an aspect, a delayed or gradual enteric release formulation comprises the use of a hydroxypropylmethylcellulose (HPMC), a microcrystalline cellulose (MCC), magnesium stearate, or a combination thereof. In an aspect, a delayed or gradual enteric release formulation comprises the use of a poly(meth)acrylate, a methacrylic acid copolymer B, a methyl methacrylate, a methacrylic acid ester, a polyvinylpyrrolidone (PVP), a PVP-K90, or a combination thereof. In an aspect, a delayed or gradual enteric release formulation comprises the use of a solid inner layer sandwiched between two outer layers; wherein said solid inner layer comprises said pharmaceutical composition and another component selected from the group consisting of a disintegrant, an exploding agent, an effervescent or any combination thereof; wherein said outer layer comprises a substantially water soluble, a crystalline polymer, or both. In an aspect, a delayed or gradual enteric release formulation comprises the use of a non-swellable diffusion matrix.

In another aspect, a delayed or gradual enteric release formulation comprises the use of a bilayer tablet or capsule which in turn comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water, depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, 2 or more, 3 or more, 4 or more, or 5 or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹² or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹² or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a pharmaceutical composition is in an anaerobic package or container. In another aspect, a pharmaceutical composition further comprises an oxygen scavenger. In one aspect, a container can be made oxygen free by, for example, incorporating into the container a built in or clipped-on oxygen-scavenging mechanism (e.g., oxygen scavenging pellets as described e.g., in U.S. Pat. No. 7,541,091). In another aspect, the container itself is made of an oxygen scavenging material (e.g., oxygen scavenging iron, as described by O2BLOCK™, or equivalents), which uses a purified and modified layered clay as a performance-enhancing carrier of oxygen-scavenging iron; the active iron is dispersed directly in the polymer. In one embodiment, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; or one or more polyethylene terephthalate homopolymers or copolymers that exhibit oxygen-scavenging activity. In one embodiment, oxygen-scavenging polymers are used to make the container itself or to coat the container, or as pellets to be added; e.g., as described in U.S. Pat. App. Pub. 20110008554, describing compositions comprising a polyester, a copolyester ether, and an oxidation catalyst, wherein the copolyester ether comprises a polyether segment comprising poly(tetramethylene-co-alkylene ether). In one embodiment, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

One challenge in treating GI infections is the high frequency of recurrence or relapse. With inadequate elimination of a infective agent (e.g., pathogenic and/or foreign bacteria), the ongoing original symptoms can return. It is known that bacteria sometimes do not divide and may live in biofilms in many wet (e.g., interior) surfaces of the body. Secondly, bacteria have spores which can be more difficult to eradicate at intermittent times of sporulation. There are also dormant forms of bacteria that can be intra- and extra-cellular where they are much more difficult to eradicate, unless the dormant form is dividing. Finally, intracellular bacteria may wait until the gut wall cell in which they are housed is shed into the gut lumen, re-infecting the flora. In alternative embodiments, the multiple or repeated bowel flora infusions of the methods of the invention can, and may be required to, kill or otherwise inactivate the viable (e.g., infective, pathogenic and/or foreign) bacteria which were protected inside the cell, biofilm and the like. In alternative embodiments, the multiple or repeated bowel flora infusions of the methods of the invention can, and may be required to, kill or otherwise inactivate bacterial cells that travel up crypts closer to the lumen, where they are shed into the faecal stream and re-infect the individual or patient.

In one aspect, a pharmaceutical composition further comprises, or is used in conjunction with, a biofilm disrupting agent. In one aspect, a biofilm disrupting agent comprises formalin. In one aspect, a biofilm disrupting agent comprises one or more enzymes selected from the group consisting of deoxyribonuclease (DNase), N-acetylcysteine, alginate lyase, and glycoside hydrolase dispersin B. In another aspect, a biofilm disrupting agent comprises one or more components selected from the group consisting of a Quorum-sensing inhibitor, a ribonucleic acid III inhibiting peptide, a Salvadora persica extract, a competence-stimulating peptide, Patulin, penicillic acid, a cathelicidin-derived peptide, a small lytic peptide, PTP-7, Nitric Oxide, neo-emulsion, ozone, a lytic bacteriophage, lactoferrin, a xylitol hydrogel, a synthetic iron chelator, curcumin, a silver nanoparticle, Acetyl-11-keto-β-boswellic acid (AKBA), sinefungin, S-adenosyl-methionine, S-adenosyl-homocysteine, a Delisea furanone, and N-sulfonyl homoserine lactones. In another aspect, a biofilm disrupting agent comprises a bismuth-thiol compound (see WO2011/097347).

A method for treating a gastrointestinal disorder in a subject in need thereof, said method comprising administering to said subject a pharmaceutically active dose of a pharmaceutical composition described herein. In one aspect, a gastrointestinal disorder being treated is Inflammatory Bowel Disease (IBD) or Irritable Bowel Syndrome (IBS). In another aspect, a gastrointestinal disorder being treated is selected from the group consisting of ulcerative colitis, Crohn's disease, indeterminate colitis, mucous colitis, collagenous colitis, Johne's disease (paratuberculosis), microscopic colitis, idiopathic inflammatory bowel disease, and antibiotic-associated colitis. In one aspect, a method further comprises removing a subject's appendix.

In one aspect, the instant application provides a method for treating IBD in a subject in need thereof, said method comprising administering a pharmaceutically active dose of an first antibiotic or probiotic to said subject to inhibit or antagonize a Fusobacterium species. In one aspect, a Fusobacterium species being inhibited is selected from the group consisting of F. nucleatum, F. necrophorum, and F. varium. In another aspect, a first probiotic comprises a Faecalibacterium species. In another aspect, a first probiotic comprises Faecalibacterium prausnitzii. In one aspect, a method further comprises administering to the subject one or more Faecalibacterium growth stimulants. In another aspect, a growth stimulant selected from the group consisting of apple pectin, N-acetyl glucosamine, cysteine, glutathione, riboflavin, and flavin. In a further aspect, a method further comprises administering a pharmaceutically active dose of a second antibiotic or second probiotic to said subject to inhibit or antagonize a Mycobacterium species. In another aspect, a Mycobacterium species being inhibited is Mycobacterium avium, ssp. paratuberculosis (MAP). In one aspect, a second probiotic comprises one or more, 2 or more, 3 or more, 4 or more, 5 or more, 6 or more, or 7 or more species selected from the anti-myco group consisting of Corynebacterium, Dietzia, Gordonia, Mycobacterium, Nocardia, Segnihparus, Skermania, Tsukamurella, Turicella, Rhodococcus, and Williamsia.

In one aspect, the instant disclosure also provides a method for treating or curing IBD in a subject in need thereof, where the method comprises removing a subject's appendix and administering to the subject a pharmaceutically active dose of a pharmaceutical composition described herein. In another aspect, a method further comprises administering to the subject a biofilm disrupting agent, an antibiotic, or both. In one aspect, a biofilm disrupting agent and an antibiotic can be administered via a single composition, sequentially, or concurrently.

In one aspect, a composition or method described herein is used and effective for treating a disorder or condition selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.

The following exemplary embodiments are listed for demonstrative purposes only:
1. A pharmaceutical composition comprising a plurality of live non-pathogenic microbes capable of inhibiting or antagonizing a Fusobacterium species.
2. The pharmaceutical composition of Embodiment 1, wherein the plurality of live non-pathogenic microbes are from a synthetic culture.
3. The pharmaceutical composition of Embodiment 1 or 2, wherein the Fusobacterium species is selected from the group consisting of F. necrophorum, F. nucleatum, F. canifelinum, F. gonidiaformans, F. mortiferum, F. naviforme, F. necrogenes, F. russii, F. ulcerans, and F. varium.
4. The pharmaceutical composition of Embodiment 1 or 2, wherein the Fusobacterium species is selected from the group consisting of F. nucleatum, F. necrophorum, and F. varium.
5. The pharmaceutical composition of Embodiment 1 or 2, wherein the plurality of live non-pathogenic microbes comprise one or more Faecalibacterium spp.
6. The pharmaceutical composition of Embodiment 1 or 2, wherein the plurality of live non-pathogenic microbes comprise Faecalibacterium prausnitzii.
7. The pharmaceutical composition of Embodiment 1 or 2, wherein the plurality of live non-pathogenic microbes comprise one or more, 2 or more, 3 or more, 4 or more, 5 or more, 6 or more, or 7 or more species or strains selected from the group consisting of Faecalibacterium prausnitzii A2-165, Faecalibacterium prausnitzii ATCC 27768, Faecalibacterium prausnitzii ATCC 27766, Faecalibacterium cf. prausnitzii KLE1255, Faecalibacterium prausnitzii L2-6, Faecalibacterium prausnitzii M21/2, Faecalibacterium prausnitzii SL3/3, Faecalibacterium sp. canine oral taxon 147, Faecalibacterium sp. DJF_VR20, Faecalibacterium sp. MC_41, Faecalibacterium sp. CAG:1138, Faecalibacterium sp. CAG:74, and Faecalibacterium sp. CAG:82.
8. The pharmaceutical composition of Embodiment 1 or 2, wherein the first plurality of live non-pathogenic microbes comprise any one of the Faecalibacterium listed in FIG. 1.
9. The pharmaceutical composition of any one of Embodiments 5 to 8, wherein the pharmaceutical composition further comprises a growth stimulant for Faecalibacterium.
10. The pharmaceutical composition of Embodiment 9, wherein the growth stimulant is selected from the group consisting of apple pectin, N-acetyl glucosamine, cysteine, glutathione, riboflavin, and flavin.
11. The pharmaceutical composition of any one of Embodiments 1 to 10, wherein the composition is in an anaerobic package or container.
12. The pharmaceutical composition of any one of Embodiments 1 to 11, wherein the composition further comprises an oxygen scavenger.
13. The pharmaceutical composition comprising a first plurality of live non-pathogenic microbes capable of inhibiting or antagonizing a Fusobacterium species and a second plurality of live non-pathogenic microbes capable of inhibiting or antagonizing a Mycobacterium species.
14. The pharmaceutical composition of Embodiment 13, wherein the first, second, or both plurality of live non-pathogenic microbes are from a synthetic culture.
15. The pharmaceutical composition of Embodiment 13 or 14, wherein the Fusobacterium species is selected from the group consisting of F. necrophorum, F. nucleatum, F. canifelinum, F. gonidiaformans, F. mortiferum, F. naviforme, F. necrogenes, F. russii, F. ulcerans, and F. varium
16. The pharmaceutical composition of Embodiment 13 or 14, wherein the Fusobacterium species is selected from the group consisting of F. nucleatum, F. necrophorum, and F. varium.
17. The pharmaceutical composition of any one of Embodiments 13 to 16, wherein the Mycobacterium species is Mycobacterium avium, ssp. paratuberculosis (MAP).
18. The pharmaceutical composition of any one of Embodiments 13 to 17, wherein the first plurality of live non-pathogenic microbes comprise a Faecalibacterium species.
19. The pharmaceutical composition of any one of Embodiments 13 to 17, wherein the first plurality of live non-pathogenic microbes comprise Faecalibacterium prausnitzii.
20. The pharmaceutical composition of any one of Embodiments 13 to 17, wherein the first plurality of live non-pathogenic microbes comprise one or more, 2 or more, 3 or more, 4 or more, 5 or more, 6 or more, or 7 or more species, isolates, or strains selected from the group consisting of Faecalibacterium prausnitzii A2-165, Faecalibacterium prausnitzii ATCC 27768, Faecalibacterium prausnitzii ATCC 27766, Faecalibacterium cf. prausnitzii KLE1255, Faecalibacterium prausnitzii L2-6, Faecalibacterium prausnitzii M21/2, Faecalibacterium prausnitzii SL3/3, Faecalibacterium sp. canine oral taxon 147, Faecalibacterium sp. DJF_VR20, Faecalibacterium sp. MC_41, Faecalibacterium sp. CAG:1138, Faecalibacterium sp. CAG:74, and Faecalibacterium sp. CAG:82.
21. The pharmaceutical composition of any one of Embodiments 13 to 17, wherein the first plurality of live non-pathogenic microbes comprise any one of the Faecalibacterium listed in FIG. 1.
22. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more species selected from the anti-myco group consisting of Corynebacterium, Dietzia, Gordonia, Mycobacterium, Nocardia, Segnihparus, Skermania, Tsukamurella, Turicella, Rhodococcus, and Williamsia.
23. The pharmaceutical composition of any one of Embodiments 13 to 21, wherein the second plurality of live non-pathogenic microbes comprise one or more, 2 or more, 3 or more, or 4 or more species selected from the group consisting of Dietzia, Gordonia, Mycobacterium, Nocardia, and Rhodococcus.
24. The pharmaceutical composition of any one of Embodiments 13 to 21, wherein the second plurality of live non-pathogenic microbes comprise Dietzia.
25. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more species selected from the group consisting of D. aerolata, D. alimentaria, D. aurantiaca, D. cerdiciphylli, D. cinnamea, D. kunjamensis, D. lutea, D. maris, D. natronolimnaea, D. papillomatosis, D. psychralcahphila, D. schimae, and D. timorensis.
26. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more Gordonia species selected from the group consisting of G. aichiensis, G. alkanivorans, G. amarae, G. amicalis, G. araii, G. bronchialis, G. defluvii, G. desulfuricans, G. effusa, G. hirstuta, G. hydrophobica, G. lacunae, G. malaquae, G. namibiensis, G. otitidis, G. paraffinivorans, G. polyisoprenivorans, G. rhizosphera, G. rubripertincta, G. shandongensis, G. sihwensis, G. sinesedis, G. soli, G. sputi, G. terrae, and G. westfalica.
27. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more Corynebacterium species selected from the group consisting of C. accolens, C. afermentans ssp. afermentans, C. ammoniagenes, C. amycolatum, C. appendicis, C. aquilae, C. argentoratense, C. atypicum, C. aurimucosum, C. auris, C. auriscanis, C. bovis, C. callunae, C. camporealensis, C. canis, C. capitovis, C. casei, C. caspium, C. ciconiae, C. confusum, C. coyleae, C. cystitidis, C. diphtheria, C. doosanense, C. durum, C. efficiens, C. falsenii, C. felinum, C. flavescens, C. freiburgense, C. freneyi, C. glaucum, C. glucuronolyticum, C. glutamicum, C. halotolerans, C. hansenii, C. imitans, C. jeikeium, C. kroppenstedtii, C. kutscheri, C. hpophiloflavum, C. lubricantis, C. macginleyi, C. marinum, C. maris, C. massiliense, C. mastitidis, C. matruchotii, C. minutissimum, C. mucifaciens, C. mustelae, C. mycetoides, C. phocae, C. pilbarense, C. pilosum, C. propinquum, C. pseudodiphtheriticum, C. pseudotuberculosis, C. pyruviciproducens, C. renale, C. resistens, C. riegelii, C. simulans, C. singular, C. sphenisci, C. spheniscorum, C. sputi, C. stationis, C. striatum, C. suicordis, C. sundsvallense, C. terpenotabidum, C. testudinoris, C. thomssenii, C. timonense, C. tuberculostearicum, C. tuscaniense, C. ulcerans, C. ulceribovis, C. urealyticum, C. ureicelerivorans, C. variabile, C. vitaeruminis, and C. xerosis.
28. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more Mycobacterium species selected from the group consisting of M. tuberculosis, M. leprae, M. avium-intracellulare, M. bovis, M. cheloiei, M. africanum, M. marinium, M. buruli, M. fortuitum, M. haemophilum, M. intracellulare, M. kansasii, M. littorals, M. malmoense, M. marianum, M. sinuae, M. szulgai, and M. ulcerans, M. avium, M. flavascens, M. lepraemurium, and M. nicroti.
29. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more Nocardia species selected from the group consisting of N. abscessus, N. acidivorans, N. africana, N. alba, N. altamirensis, N. amamiensis, N. anaemiae, N. aobensis, N. araoensis, N. arthritidis, N. asiatica, N. asteroides, N. beijingensis, N. blacklockiae, N. brasiliensis, N. brevicatena, N. caishijiensis, N. calitrisensis, N. carnea, N. cerradoensis, N. concava, N. coubleae, N. crassostreae, N. cummidelens, N. cyriacigeorgica, N. elegans, N. exalbida, N. farcinica, N. flavorosea, N. fluminea, N. gamkensis, N. harensis, N. higoensis, N. ignorata, N. inohanensis, N. iowensis, N. jejuensis, N. jiangxiensis, N. jinanensis, N. kruczakiae, N. lijiangensis, N. mexicana, N. miyunensis, N. neocaledoniensis, N. niigataensis, N. ninae, N. nova, N. otitidiscaviarum, N. paucivorans, N. pigrifrangens, N. pneumoniae, N. polyresistens, N. pseudobrasiliensis, N. pseudovaccinii, N. puris, N. salmonicida, N. seriolae, N. shimofusensis, N. sienata, N. soli, N. speluncae, N. takedensis, N. tenerifensis, N. terpenica, N. testacea, N. thailandica, N. transvalensis, N. uniformis, N. vaccinii, N. vermiculata, N. veterana, N. vinacea, N. wallacei, N. xishanensis, and N. yamanashiensis.
30. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more Rhodococcus species selected from the group consisting of R. aurantiacus, R. aetherivorans, R. baikonurensis, R. coprophilus, R. corynebacterioides, R. equi, R. erythropolis, R. fascians, R. globerulus, R. gordoniae, R. imtechensis, R. jostii, R. koreensis, R. kroppenstedtii, R. kunmingensis, R. kyotonensis, R. maanshanensis, R. marinonascens, R. opacus, R. percolatus, R. phenolicus, R. pyridinivorans, R. qingshengii, R. rhodnii, R. rhodochrous, R. ruber, R. triatomae, R. tukisamuensis, R. wratislaviensis, R. yunnanensis, and R. zopfii.
31. The pharmaceutical composition of any one of Embodiments 13 to 21, wherein the second plurality of live non-pathogenic microbes comprise one or more, 2 or more, 3 or more, 4 or more, 5 or more, 6 or more, or all 7 species selected from the group consisting of Skermania piniformis, Williamsia deligens, Williamsia serinedens, Williamsia maris, Williamsia marianensis, Williamsia muralis, and Williamsia faeni.
32. The pharmaceutical composition of any one of Embodiments 13 to 21, wherein the second plurality of live non-pathogenic microbes comprise one or more, 2 or more, 3 or more, 4 or more, 5 or more, 6 or more, or 7 or more Tsukamurella species selected from the group consisting of T. paurometabola, T. spumae, T. inchonensis, T. sunchonensis, T. pseudospumae, T. spongiae, T. pulmonis, T. tyrosinosolvens, and T. strandjordii.
33. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes is selected from the group consisting of 10³ cfu to 10¹⁴ cfu, 10⁴ cfu to 10¹⁴ cru 10⁵ cfu to 10¹⁴ cfu, 10⁶ cfu to 10¹⁴ cfu, 10⁷ cfu to 10¹⁴ cfu, 10⁸ cfu to 10¹⁴ cfu 10⁴ cfu to 10¹³ cfu, 10⁵ cfu to 10¹² cfu, 10⁶ cfu to 10¹¹ cfu, 10⁷ cfu to 10¹⁰ cfu, 10⁸ cfu to 10⁹ cfu, 10³ cfu to 10¹³ cfu, 10³ cfu to 10¹² cfu, 10³ cfu to 10¹¹ cfu, 10³ cfu to 10¹⁰ cfu, 10³ cfu to 10⁹ cfu, 10³ cfu to 10⁸ cfu, 10³ cfu to 10⁷ cfu, 10³ cfu to 10⁶ cfu, 10³ cfu to 10⁵ cfu, and 10³ cfu to 10⁴ cfu.
34. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes is selected from the group consisting of from 10⁸ cfu to 10¹⁴ cfu, 10⁹ cfu to 10¹³ cfu, from 10¹⁰ cfu to 10¹² cfu, from 10⁹ cfu to 10¹⁴ cfu, from 10⁹ cfu to 10¹² from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu.
35. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes comprises at least 10³, 10⁴, 10⁵, 10⁶, 10⁷, 10⁸, 10⁹, 10¹⁰, 10¹¹, 10¹², 10¹³, 10¹⁴, or 10¹⁵ cfu.
36. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes comprises at most 10³, 10⁴, 10⁵, 10⁶, 10⁷, 10⁸, 10⁹, 10¹⁰, 10¹¹, 10¹², 10¹³, 10¹⁴, or 10¹⁵ cfu.
37. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes comprises a cell count selected from the group consisting of 10³ to 10¹⁴, 10⁴ to 10¹⁴, 10⁵ to 10¹⁴ 10⁶ to 10¹⁴, 10⁷ to 10¹⁴, 10⁸ to 10¹⁴, 10⁴ to 10¹³, 10⁵ to 10¹², 10⁶ to 10¹¹, 10⁷ to 10¹⁰, 10⁸ to 10⁹, 10³ to 10¹³, 10³ to 10¹², 10³ to 10¹¹, 10³ to 10¹⁰, 10³ to 10⁹, 10³ to 10⁸, 10³ to 10⁷, 10³ to 10⁶, 10³ to 10⁵, and 10³ to 10⁴.
38. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes comprises a cell count selected from the group consisting of from 10⁸ to 10¹⁴, 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴.
39. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes comprises a cell count of at least 10³, 10⁴, 10⁵, 10⁶, 10⁷, 10⁸, 10⁹, 10¹⁰, 10¹¹, 10¹², 10¹³, 10¹⁴, or 10¹⁵.
40. The pharmaceutical composition of any one of Embodiments 13 to 32, wherein a pharmacologically active dose of the first plurality of live non-pathogenic microbes, the second plurality of live non-pathogenic microbes, or a combination of the first and the second plurality of live non-pathogenic microbes comprises a cell count of at most 10³, 10⁴, 10⁵, 10⁶, 10⁷, 10⁸, 10⁹, 10¹⁰, 10¹¹, 10¹², 10¹³, 10¹⁴, or 10¹⁵,
41. The pharmaceutical composition of any one of Embodiments 13 to 40, wherein the pharmaceutical composition is in a liquid, frozen, freeze-dried, spray-dried, lyophilized, or powder form.
42. The pharmaceutical composition of any one of Embodiments 13 to 41, wherein the pharmaceutical composition comprises an excipient, a saline, a buffer, a buffering agent, or a fluid-glucose-cellobiose agar (RGCA) media.
43. The pharmaceutical composition of any one of Embodiments 13 to 42, wherein the pharmaceutical composition is formulated as an enteric coated capsule or microcapsule, an acid-resistant capsule or microcapsule, an enteric coated tablet, an acid-resistant tablet, a powder suitable for reconstitution, a naso-duodenal infusion, or for delivery in the form of an enema or a colonoscopic infusion.
44. The pharmaceutical composition of any one of Embodiments 13 to 42, wherein the pharmaceutical composition is formulated as a delayed or gradual enteric release form.
45. The pharmaceutical composition of Embodiment 44, wherein the delayed or gradual enteric release formulation comprises the use of cellulose acetate, polyethylene glycerol, or both.
46. The pharmaceutical composition of Embodiment 44, wherein the delayed or gradual enteric release formulation comprises the use of a hydroxypropylmethylcellulose (HPMC), a microcrystalline cellulose (MCC), magnesium stearate, or a combination thereof.
47. The pharmaceutical composition of Embodiment 44, wherein the delayed or gradual enteric release formulation comprises the use of a poly(meth)acrylate, a methacrylic acid copolymer B, a methyl methacrylate, a methacrylic acid ester, a polyvinylpyrrolidone (PVP), a PVP-K90, or a combination thereof.
48. The pharmaceutical composition of Embodiment 44, wherein the delayed or gradual enteric release formulation comprises the use of a solid inner layer sandwiched between two outer layers; wherein the solid inner layer comprises the pharmaceutical composition and another component selected from the group consisting of a disintegrant, an exploding agent, an effervescent or any combination thereof; wherein the outer layer comprises a substantially water soluble, a crystalline polymer, or both.
49. The pharmaceutical composition of Embodiment 44, wherein the delayed or gradual enteric release formulation comprises the use of a non-swellable diffusion matrix.
50. The pharmaceutical composition of Embodiment 44, wherein the delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both.
51. The pharmaceutical composition of Embodiment 44, wherein the delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof
52. The pharmaceutical composition of Embodiment 44, wherein the delayed or gradual enteric release formulation comprises the use of a microenvironment pH modifier.
53. The pharmaceutical composition of any one of Embodiments 13 to 42, wherein the composition is administered together with a food, a liquid beverage, a food additive, a dairy-based product, a soy-based product or a derivative thereof, a jelly, or a yogurt.
54. The pharmaceutical composition of any one of Embodiments 13 to 53, wherein the first plurality of live non-pathogenic microbes, the second plurality of live non-pathogenic microbes, or both the first and the second plurality of live non-pathogenic microbes combined are in an anaerobic package or container.
55. The pharmaceutical composition of any one of Embodiments 13 to 53, wherein the first plurality of live non-pathogenic microbes, the second plurality of live non-pathogenic microbes, or both the first and the second plurality of live non-pathogenic microbes combined are in an aerobic package or container.
56. The pharmaceutical composition of any one of Embodiments 1 to 55, wherein the pharmaceutical composition comprises a cryoprotectant.
57. The pharmaceutical composition of Embodiment 56, wherein the cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.
58. The pharmaceutical composition of any one of Embodiments 1 to 57, wherein the pharmaceutical composition further comprises an acid suppressant, an antacid, an H2 antagonist, a proton pump inhibitor or a combination thereof.
59. The pharmaceutical composition of any one of Embodiments 1 to 58, wherein the pharmaceutical composition further comprises a fecal microbiota preparation.
60. A pharmaceutical composition comprising a fecal microbiota from a single donor subject administered with one or more Faecalibacterium species or one or more growth stimulating agents for at least one Faecalibacterium species, wherein the fecal microbiota comprises an elevated level of the at least one Faecalibacterium species relative to a control fecal microbiota from the same donor subject not administered with the one or more growth stimulating agents.
61. The pharmaceutical composition of Embodiment 60, wherein the donor subject ingests the one or more Faecalibacterium species or one or more growth stimulating agents.
62. The pharmaceutical composition of Embodiment 60 or 61, wherein the one or more growth stimulating agents are selected from the group consisting of apple pectin, N-acetyl glucosamine, cysteine, glutathione, riboflavin, and Flavin.
63. The pharmaceutical composition of any one of Embodiments 60 to 62, wherein the fecal microbiota is from a fecal sample collected at least about 1, 2, 3, 4, 5, 6, 7, 8, 9, 10, 11, 12, 13, 14, 18, 20, 24, 28, 30, or 36 hours after the administration of the growth stimulating agent.
64. The pharmaceutical composition of any one of Embodiments 60 to 62, wherein the fecal microbiota is from a fecal sample collected at least about 1, 2, 3, 4, 5, or 6 days after the administrating of the growth stimulating agent.
65. The pharmaceutical composition of any one of Embodiments 60 to 62, wherein the one or more growth stimulating agents are administered to the donor subject for more than about 1, 2, 3, 4, 5, 6, 7, 8, 9, 10, 11, 12, 13, or 14 days. 66. The pharmaceutical composition of any one of Embodiments 60 to 62, wherein the one or more growth stimulating agents are administered to the donor subject for more than about 1, 2, 3, 4, 5, 6, 7, 8, 9, 10, 11, 12, 13, or 14 weeks.
67. The pharmaceutical composition of any one of Embodiments 60 to 66, wherein the one or more growth stimulating agents are administered to the donor subject at least 1, 2, 3, 4, 5, 6, 7, 8, 9, or 10 times daily.
68. A pharmaceutical composition, comprising a non-synthetic fecal microbiota, wherein the non-synthetic fecal microbiota comprises an elevated level of at least one Faecalibacterium species relative to a control fecal microbiota from a normal healthy donor.
69. A pharmaceutical composition comprising a non-selected fecal microbiota, wherein the non-selected fecal microbiota comprises an elevated level of at least one Faecalibacterium species relative to a control non-selected fecal microbiota from a normal healthy donor.
70. A pharmaceutical composition comprising an untreated, non-synthetic fecal microbiota from a single donor subject, wherein the untreated, non-synthetic fecal microbiota comprises an elevated level of at least one Faecalibacterium species relative to a control fecal microbiota from a normal healthy donor.
71. The pharmaceutical composition of any one of Embodiments 60 to 70, wherein the elevated level is selected from the group consisting of 1.5-fold or more, 2-fold or more, 2.5-fold or more, 3-fold or more, 3.5-fold or more, 4-fold or more, 5-fold or more, 10-fold or more, 50-fold or more, 100-fold or more, 1000-fold or more, and 10,000-fold or more.
72. The pharmaceutical composition of any one of Embodiments 60 to 70, wherein the elevated level is selected from the group consisting of at least 10% more, at least 15% more, at least 20% more, at least 25% more, at least 30% more, at least 40% more, at least 50% more, at least 60% more, at least 70% more, at least 80% more, at least 90% more, at least 100% more, at least 150% more, at least 200% more, at least 250% more, at least 300% more, at least 350% more, at least 400% more, at least 450% more, at least 500% more, at least 600% more, at least 700% more, and at least 800% more.
73. The pharmaceutical composition of any one of Embodiments 59 to 72, wherein the fecal microbiota or the fecal microbiota preparation comprises a donor's entire or substantially complete microbiota.
74. The pharmaceutical composition of any one of Embodiments 59 to 72, wherein the fecal microbiota or the fecal microbiota preparation comprises a non-selected fecal microbiota.
75. The pharmaceutical composition of any one of Embodiments 59 to 72, wherein the fecal microbiota or the fecal microbiota preparation comprises an isolated or purified population of live non-pathogenic fecal bacteria.
76. The pharmaceutical composition of any one of Embodiments 59 to 72, wherein the preparation of the fecal microbiota or the fecal microbiota preparation involves a treatment selected from the group consisting of ethanol treatment, detergent treatment, heat treatment, irradiation, and sonication, or a combination thereof
77. The pharmaceutical composition of any one of Embodiments 59 to 72, wherein the preparation of the fecal microbiota or the fecal microbiota preparation involves no treatment selected from the group consisting of ethanol treatment, detergent treatment, heat treatment, irradiation, and sonication.
78. The pharmaceutical composition of any one of Embodiments 59 to 72, wherein the preparation of the fecal microbiota or preparation involves a separation step selected from the group consisting of filtering, sieving, density gradients, filtration, chromatography, and a combination thereof.
79. The pharmaceutical composition of any one of Embodiments 59 to 72, wherein the fecal microbiota or the fecal microbiota preparation does not require one or more separation steps selected from the group consisting of filtering, sieving, density gradients, filtration, and chromatography.
80. The pharmaceutical composition of any one of Embodiments 59 to 72, wherein the fecal microbiota or the fecal microbiota preparation is substantially free of non-living matter.
81. The pharmaceutical composition of any one of Embodiments 59 to 72, wherein the fecal microbiota or the fecal microbiota preparation is substantially free of acellular material selected from the group consisting of residual fiber, DNA, viral coat material, and non-viable material.
82. The pharmaceutical composition of any one of Embodiments 59 to 72, wherein the fecal microbiota or the fecal microbiota preparation is substantially free of eukaryotic cells from the fecal microbiota's donor.
83. The pharmaceutical composition of Embodiment 59, wherein the fecal microbiota preparation is from reconstituted fecal material.
84. The pharmaceutical composition of Embodiment 59, wherein the fecal microbiota preparation is from synthetic fecal material.
85. The pharmaceutical composition of any one of Embodiments 59 to 72, wherein the fecal microbiota or the fecal microbiota preparation comprises no antibiotic resistant population.
86. The pharmaceutical composition of Embodiment 59, wherein the fecal microbiota preparation comprises a preparation of viable flora in proportional content that resembles a normal healthy human fecal flora.
87. The pharmaceutical composition of any one of Embodiments 59 to 72, wherein the fecal microbiota or the fecal microbiota preparation comprises bacteria from at least 2, 3, 4, 5, 6, 7, 8, 9, 10, 12, 15, 18, or 20 different families.
88. The pharmaceutical composition of any one of Embodiments 59 to 72, wherein the fecal microbiota or the fecal microbiota preparation comprises bacteria from at least 2, 3, 4, 5, 6, 7, 8, 9, 10, 12, 15, 18, 20, 23, 25, 27, 30, 32, 35, 38, or 40 different genera.
89. The pharmaceutical composition of any one of Embodiments 59 to 72, wherein the fecal microbiota or the fecal microbiota preparation has a Shannon Diversity Index of 0.4-5.0 at the family, genus, or species level.
90. The pharmaceutical composition of any one of Embodiments 59 to 72, wherein the fecal microbiota or the fecal microbiota preparation has at least about 20%, 30%, 40%, 50%, 60%, 70%, 80%, 85%, 90%, 95%, 99%, or 99.5% microbes in a spore form.
91. The pharmaceutical composition of any one of Embodiments 59 to 72, wherein the fecal microbiota or the fecal microbiota preparation has at least about 20%, 30%, 40%, 50%, 60%, 70%, 80%, 85%, 90%, 95%, 99%, or 99.5% microbes in a non-spore form.
92. The pharmaceutical composition of any one of Embodiments 59 to 72, wherein the fecal microbiota or the fecal microbiota preparation is further supplemented or enhanced with one or more, 2 or more, 3 or more, 4 or more, or 5 or more cultured fecal microorganisms.
93. The pharmaceutical composition of Embodiment 92, wherein the one or more, one or more, 2 or more, 3 or more, 4 or more, or 5 or more fecal microorganisms are in a spore form.
94. The pharmaceutical composition of Embodiment 92, wherein the one or more, 2 or more, 3 or more, 4 or more, or 5 or more fecal microorganisms are selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella.
95. The pharmaceutical composition of Embodiment 92, wherein the one or more, 2 or more, 3 or more, 4 or more, or 5 or more fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.
96. The pharmaceutical composition of any one of Embodiments 59 to 72, wherein the fecal microbiota or the fecal microbiota preparation lacks at least one, at least 2, at least 3, or at least 4 genera of bacteria selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella.
97. The pharmaceutical composition of any one of Embodiments 59 to 72, wherein the fecal microbiota or the fecal microbiota preparation lacks at least one, at least 2, at least 3, or at least 4 microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella orahs, Prevotella ruminicola, Odoribacter splanchnicus, and Desuifomonas pigra.
98. The pharmaceutical composition of any one of the preceding Embodiments, wherein the pharmaceutical composition further comprises a biofilm disrupting agent.
99. The pharmaceutical composition of Embodiment 98, wherein the biofilm disrupting agent comprises one or more enzymes selected from the group consisting of deoxyribonuclease (DNase), N-acetylcysteine, alginate lyase, and glycoside hydrolase dispersin B.
100. The pharmaceutical composition of Embodiment 98, wherein the biofilm disrupting agent comprises one or more components selected from the group consisting of Quorum-sensing inhibitors, a ribonucleic acid III inhibiting peptide, a Salvadora persica extract, a competence-stimulating peptide, Patulin, penicillic acid, a cathelicidin-derived peptide, a small lytic peptide, PTP-7, Nitric Oxide, neo-emulsion, ozone, a lytic bacteriophage, lactoferrin, a xylitol hydrogel, a synthetic iron chelator, curcumin, a silver nanoparticle, Acetyl-11-keto-O-boswellic acid (AKBA), sinefungin, S-adenosyl-methionine, S-adenosyl-homocysteine, a Delisea furanone, and N-sulfonyl homoserine lactones.
101. A method for treating a gastrointestinal disorder in a subject in need thereof, the method comprising administering to the subject a pharmaceutically active dose of the pharmaceutical composition of any one of Embodiments 1 to 100.
102. The method of Embodiment 101, wherein the gastrointestinal disorder is Inflammatory Bowel Disease (IBD) or Irritable Bowel Syndrome (MS).
103. The method of Embodiment 101, wherein the gastrointestinal disorder is selected from the group consisting of ulcerative colitis, Crohn's disease, indeterminate colitis, mucous colitis, collagenous colitis, Johne's disease (paratuberculosis), microscopic colitis, idiopathic inflammatory bowel disease, and antibiotic-associated colitis, ischemic colitis, diversion colitis, pseudomembranous colitis, and lymphocytic colitis.
104. The method of Embodiment 101, wherein the gastrointestinal disorder is ulcerative colitis.
105. The method of Embodiment 104, wherein the ulcerative colitis is selected from the group consisting of ulcerative proctitis, proctosigmoiditis, left-sided colitis, and pan-ulcerative colitis.
106. The method of Embodiment 104, wherein the method reduces the ulcerative colitis disease activity index (UCDAI) of the patient by at least 10%, 20%, 30%, 50%, 60%, 70%, 80%, or 90% after 8 weeks of treatment.
107. The method of Embodiment 104, wherein the administration is on a daily basis.
108. The method of Embodiment 104, wherein the administration lasts at least 1, 2, 3, 4, 5, 6, 7, 8, 9, or 10 weeks.
109. The method of Embodiment 104, wherein the dose is administered at least once daily for at least 2, 3, 4, 5, 6, 7, 8, 9, 10, 11, 12, 13, 14, or 15 consecutive days.
110. The method of Embodiment 104, wherein the dose is administered at least once daily for at least 1, 2, 3, 4, 5, 6, 7, 8, 9, 10, 11, or 12 consecutive weeks.
111. The method of Embodiment 104, wherein the dose is administered at least once daily for at most 3, 4, 5, 6, 7, 8, 9, 10, 11, 12, 13, 14, 15, 16, 17, 18, 19, or 20 consecutive days.
112. The method of Embodiment 104, wherein the dose is administered at least once daily for at most 1, 2, 3, 4, 5, 6, 7, 8, 9, 10, 11, or 12 consecutive weeks.
113. The method of Embodiment 104, wherein the dose is administered at least twice daily for at least two consecutive days.
114. The method of Embodiment 104, wherein the dose is administered at least twice daily for at least 3, 4, 5, 6, 7, 8, 9, 10, 11, 12, 13, 14, or 15 consecutive days.
115. The method of Embodiment 104, wherein the dose is administered at least twice daily for at least 1, 2, 3, 4, 5, 6, 7, 8, 9, 10, 11, or 12 consecutive weeks.
116. The method of Embodiment 104, wherein the dose is administered at least twice daily for at most 3, 4, 5, 6, 7, 8, 9, 10, 11, 12, 13, 14, 15, 16, 17, 18, 19, or 20 consecutive days.
117. The method of Embodiment 104, wherein the dose is administered at least twice daily for at most 1, 2, 3, 4, 5, 6, 7, 8, 9, 10, 11, or 12 consecutive weeks.
118. The method of Embodiment 104, wherein the dose is administered at least three times daily for at least one day.
119. The method of Embodiment 104, wherein the dose is administered at least three times daily for at least 2, 3, 4, 5, 6, 7, 8, 9, 10, 11, 12, 13, 14, or 15 consecutive days.
120. The method of Embodiment 104, wherein the dose is administered at least three times daily for at most 2, 3, 4, 5, 6, 7, 8, 9, 10, 11, 12, 13, 14, or 15 consecutive days.
121. The method of any one of preceding Embodiments, wherein the method comprises a first dosing schedule followed by a second dosing schedule.
122. The method of Embodiment 121, wherein the second dosing schedule comprises a maintenance dose lower or equal to the dose of the first dosing schedule.
123. The method of Embodiment 121, wherein the second dosing schedule lasts for at least about 2, 4, 6, 8, 10, 12, 18, 24, 36, 48, 72, or 96 months.
124. The method of Embodiment 121, wherein the second dosing schedule lasts permanently.
125. The method of Embodiment 121, wherein the interval between the first and second dosing schedules is at least about 1, 2, 3, 4, 5, 6, 7, 8, 9, 10, 11, or 12 weeks.
126. The method of Embodiment 121, wherein the second dosing schedule is a continuous dosing schedule.
127. The method of Embodiment 121, wherein the second dosing schedule is an intermittent dosing schedule.
128. The method of Embodiment 121, wherein the intermittent dosing schedule comprises a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days.
129. The method of any one of preceding Embodiments, wherein the administering comprises administering orally, by nasal duodenal, by enema, or via rectal suppository.
130. The method of any one of preceding Embodiments, wherein the method eliminates or reduces gastrointestinal dysbiosis.
131. The method of any one of preceding Embodiments, wherein the method increases bacterial diversity in the subject's gastrointestinal tract.
132. The method of any one of preceding Embodiments, wherein the subject is pretreated with an antibiotic prior to administration of the composition.
133. The method of Embodiment 132, wherein the antibiotic is selected from the group consisting of amoxicillin, tetracycline, metronidazole, rifabutin, clarithromycin, clofazimine, vancomycin, rifampicin, nitroimidazole, chloramphenicol, and a combination thereof; and optionally the antibiotic comprises an antibiotic combination regimen having amoxicillin, tetracycline, and metronidazole (ATM).
134. The method of Embodiment 132, wherein the antibiotic is selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.
135. The method of any one of preceding Embodiments, wherein the subject is pretreated with an anti-inflammatory drug prior to administration of the composition.
136. The method of any one of preceding Embodiments, wherein the method eliminates or reduces one or more, 2 or more, 3 or more, 4 or more symptoms selected from the group consisting of diarrhoea, cramp, tenesmus, weight loss, bleeding, loss of appetite, abdominal pain, fever, fatigue, anaemia, inflammation, and micro-ulcers.
137. The method of any one of preceding Embodiments, wherein the method further comprises administering a 5-aminosalicylic acid agent, a corticosteroid, an immunosuppressant, or a combination thereof.
138. The method of any one of preceding Embodiments, wherein the method further comprises administering 5-aminosalicylic acid or a derivative thereof, sulfasalazine or a derivative thereof, or a combination thereof
139. The method of any one of preceding Embodiments, wherein a biofilm disrupting agent is administered prior to the administration of the pharmaceutical composition.
140. The method of any one of preceding Embodiments, wherein a biofilm disrupting agent is administered following the administration of the pharmaceutical composition.
141. The method of any one of preceding Embodiments, wherein a biofilm disrupting agent is administered concurrently with the pharmaceutical composition.
142. The method of any one of preceding Embodiments, wherein the method further comprises removing the subject's appendix.
143. The method for treating a disorder or condition in a subject in need thereof, said method comprising administering to the subject a pharmaceutically active dose of the pharmaceutical composition of any one of Embodiments 1 to 100 effective for treating the disorder or condition, wherein the disorder or condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.
144. A method for treating IBD in a subject in need thereof, the method comprising administering a pharmaceutically active dose of an first antibiotic or probiotic to the subject to inhibit or antagonize a Fusobacterium species.
145. The method of Embodiment 144, wherein the Fusobacterium species is selected from the group consisting of F. nucleatum, F. necrophorum, and F. varium.
146. The method of Embodiment 144 or 145, wherein the first probiotic comprises a Faecalibacterium species.
147. The method of Embodiment 144 or 145, wherein the first probiotic comprises Faecalibacterium prausnitzii
148. The method of Embodiment 144, wherein the method further administering a pharmaceutically active dose of an second antibiotic or probiotic to the subject to inhibit or antagonize a Mycobacterium species.
149. The method of Embodiment 148, wherein the Mycobacterium species is Mycobacterium avium, ssp. paratuberculosis (MAP).
150. The method of Embodiment 148 or 149, wherein the probiotic comprises one or more, 2 or more, 3 or more, 4 or more, 5 or more, 6 or more, or 7 or more species selected from the anti-myco group consisting of Corynebacterium, Dietzia, Gordonia, Mycobacterium, Nocardia, Segnihparus, Skermania, Tsukamurella, Turicella, Rhodococcus, and Williamsia.
151. A method for treating or curing IBD in a subject in need thereof, the method comprising: removing the subject's appendix, administering to the subject a biofilm disrupting agent, administering to the subject an antibiotic, and administering to the subject a pharmaceutically active dose of the pharmaceutical composition of any one of Embodiments 1 to 100.
152. A method comprising administering to a subject a growth stimulating agent for a Faecalibacterium species and collecting a fecal sample from the subject for preparing a fecal microbiota composition.
153. The method of Embodiment 152, wherein the fecal microbiota composition comprises an elevated level of the Faecalibacterium species relative to a control fecal microbiota composition from the same subject without taking the growth stimulating agent.
154. The method of Embodiment 152 or 153, wherein the subject orally ingests the growth stimulating agent.
155. The method of any one of Embodiments 152 to 154, wherein the growth stimulating agent is selected from the group consisting of apple pectin, N-acetyl glucosamine, cysteine, glutathione, riboflavin, and Flavin.
156. The method of any one of Embodiments 152 to 155, wherein the fecal sample is collected at least about 1, 2, 3, 4, 5, 6, 7, 8, 9, 10, 11, 12, 13, 14, 18, 20, 24, 28, 30, or 36 hours after the administrating of the growth stimulating agent.
157. The method of any one of Embodiments 152 to 155, wherein the fecal sample is collected at least about 1, 2, 3, 4, 5, or 6 days after the administrating of the growth stimulating agent.
158. The method of any one of Embodiments 152 to 155, wherein the growth stimulating agent is administered to the subject for more than about 1, 2, 3, 4, 5, 6, 7, 8, 9, 10, 11, 12, 13, or 14 days before collecting the fecal sample.
159. The method of any one of Embodiments 152 to 155, wherein the growth stimulating agent is administered to the subject for more than 1, 2, 3, 4, 5, 6, 7, 8, 9, 10, 11, 12, 13, or 14 weeks before collecting the fecal sample.
160. The method of any one of Embodiments 152 to 155, wherein the growth stimulating agent is administered to the subject at least 1, 2, 3, 4, 5, 6, 7, 8, 9, or 10 times daily.
161. The method of any one of Embodiments 152 to 155, wherein the fecal microbiota composition comprises 1.5-fold or more, 2-fold or more, 2.5-fold or more, 3-fold or more, 3.5-fold or more, 4-fold or more, 5-fold or more, 10-fold or more, 50-fold or more, 100-fold or more, 1000-fold or more, or 10,000-fold or more of Faecalibacterium compared to a control fecal microbiota composition from the same subject without the administering of the growth stimulating agent.
162. The method of any one of Embodiments 152 to 155, wherein the fecal microbiota composition comprises at least 10% more, 15% more, 20% more, 25% more, 30% more, 40% more, 50% more, 60% more, 70% more, 80% more, 90% more, 100% more, 150% more, 200% more, 250% more, 300% more, 350% more, 400% more, 450% more, 500% more, 600% more, 700% more, or 800% more Faecalibacterium relative to a control fecal microbiota composition from the same subject without the administering of the growth stimulating agent.
163. The method of any one of Embodiments 152 to 155, further comprising producing a pharmaceutical composition from the fecal microbiota.
164. The method of Embodiment 163, wherein the pharmaceutical composition comprises between about 10³ and about 10¹³, between about 10⁴ and about 10¹², between about 10⁵ and about 10¹¹, between about 10⁶ and about 10¹⁰, between about 10⁷ and about 10⁹, or between about 10⁷ and about 10⁸ viable Faecalibacterium.
165. The method of Embodiment 163, wherein the production of the pharmaceutical composition does not go through a treatment selected from the group consisting of ethanol treatment, detergent treatment, heat treatment, irradiation, and sonication.
166. The method of Embodiment 163, wherein the production of the pharmaceutical composition comprises a treatment step selected from the group consisting of ethanol treatment, detergent treatment, heat treatment, irradiation, and sonication.
167. The method of Embodiment 163, wherein the production of the pharmaceutical composition comprises a separation step selected from the group consisting of density gradients, filtration, and chromatography.
168. The method of Embodiment 163, wherein the production of the pharmaceutical composition does not comprise a separation step selected from the group consisting of density gradients, filtration, and chromatography.

### EXAMPLES

### Example 1. Treatment of Ulcerative Colitis

Patients with an HBI of ≥7 are selected for treatment by administering a fecal microbiota composition if at least one or more of the following standards is met: male and female patients aged more than 18 years; diagnosis of ulcerative colitis (UC) established by previous colonoscopy, with consistent histology and clinical course; UC involving at least the rectosigmoid region; activity confirmed by colonoscopy at the beginning of the study; mild-to-moderate relapsing UC, defined as a ulcerative colitis disease activity index (UCDAI) score ranging from three to eight; symptoms (relapsing episodes) for less than 4 weeks before study entry; a minimum endoscopic score of three on the UCDAI at screening (mucosal appearance); use of oral 5-aminosalicylic acid (5-ASA) at least 4 weeks before study entry at a stable dose (mesalazine at least 1.6 g/day or balsalazide at least 4.5 g/day) and/or use of azathioprine (at least 1.5 mg/kg/day) or 6-mercaptopurine (at least 1 mg/kg/day) at least 3 months before study entry at a stable dose.

The following criteria are used to exclude certain patients: the presence of Crohn's disease or pouchitis; a UCDAI score greater than eight (the need for emergency surgery or the presence of severe disease); use of oral steroids within the last 4 weeks before study entry; use of antibiotics within the last 2 weeks before study entry; change in dose of oral 5-ASA within the last 4 weeks before study entry and throughout the 8-week study period or a change in dose of oral 6-mercaptopurine and azathioprine drugs within the last 3 months before the study; use of rectal 5-ASA or steroids within 1 week before entering the study or throughout the 8-week study period; use of probiotic preparations either prescribed or over-the-counter within 2 weeks before study entry; use of NSAIDs (non-steroidal anti-inflammatory drug) for 1 week before and throughout the 8-week study period.

Patients cease taking all conventional ulcerative colitis treatments at least one week prior to the administration of a fecal microbiota composition. Additionally, patients are given an anti-inflammatory drug (e.g., mesalazine) and/or one or more antibiotics prior to administration of the mixture composition. The mixture composition may also contain an acid suppressant, an antacid (e.g., aluminum hydroxide, magnesium hydroxide, simethicone antacids), an H2 antagonist (e.g., ranitidine), a proton pump inhibitor (e.g., omeprazole), or a combination thereof. A pharmaceutical composition described here is administered orally once a day. Each patient receives a constant dose; however, the volume of the dose may depend on body weight.

Patients are evaluated for abdominal symptoms and extraintestinal manifestations 3 days, 1 week, 1 month, 3 months, 6 months, 9 months, and 12 months after administration

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entirety.

### Example 2. A Faecalibacterium-Enriched Fecal Microbiota-Based Therapy of Pancolitis

An 8 year old female patient, presenting a two-year history of pancolitis, is treated with a Faecalibacterium-enriched fecal microbiota-based therapy. Previous treatments based on steroids, immunosuppressants and anti-inflammatory agents largely failed for this patient. The patient continued to pass 7-8 bloody stools every day with mucus and marked urgency and abdominal pain until she is given fecal microbiota transplantation (FMT).

For the FMT, she is pre-treated with rifaximin, metronidazole and vancomycin and remained on azathioprine (Imuran) and Budesonide. After achieving some improvement in bowel function, she undergoes a 5 day fresh donor stool-derived full spectrum microbiota FMT. This is followed with daily home enemas with her mother as the donor since late December 2013. The patient achieves formed stool, is able to return to school, and undergoes a growth spurt. Steroids are reduced, as is the Imuran. However, daily enemas are mostly required to prevent relapse. This situation continued with over 650 home donor enemas carried out, mostly 6-7 times every 7 days, with stool still mostly soft, mild urgency, and visible mucus.

To further improve the patient's conditions, Faecalibacterium growth stimulants are given to both the stool donor (mother) and the patient. The donor [mother] stool is tested for F. prausnitzii content before and after the mother is given a daily oral feeding of apple pectin (and later N-acetyl-glucosamine). The endogenous F. prausnitzii content of donor stool rises by approximately 10⁴ after the donor is supplemented with apple pectin and N-acetyl-glucosamine. Within 7 days of this supplementation, the recipient daughter's stool quality dramatically changes to more firm, becomes less frequent, without mucus, urgency disappears, and the enema frequency is reduced to 4-5 enemas/week. After this improvement, the daughter is also given the same oral supplements of apple pectin or N-acetyl-glucosamine, resulting in some further improvement in stool formation and the ability to cease Budesonide. The daughter is continuing with home-enema FMT but she has much more formed stools and generally requires to have enemas at a further reduced frequency (<4 enemas every 7 days).

### Example 3: Faecalibacterium-Enriching Diet for Fecal Microbiome-Based Treatment of a Colitis Patient

A 24-year-old female patient develops severe colitis during an international trip. Her bloody diarrhea is severe and her hemoglobin drops down to about 3.0, a low level generally considered to be potentially fatal. She is treated with multiple blood transfusions and anti-colitis treatments.

Recovering partially, she returns to Australia, where she is admitted to hospital and it is found to be still bleeding quite briskly from her severe pancolitis. Anti-inflammatory agents, including steroids, 6-MP, 5-ASA compounds, as well as intravenous fluids, are administered, and iron infusions are carried out. She seeks a second opinion of treatment options as her hemoglobin remains around 9-10 in spite of maximal therapies with a colectomy planned.

At repeat colonoscopy she is found to have ongoing patchy colitis with only some areas of healing, but mostly oozing blood on contact with the mucosa by the colonoscope. Cultures are negative for C. difficile and other pathogens, and she continues ooze blood from the mucosa in association with loss of visible vessels across the entire colon, but the terminal ileum is not affected.

She begins to be treated with fecal microbiome-based therapy to prevent a colectomy. Her symptoms improve quite markedly and the hemoglobin rises to between 10 and 11. However, she cannot return to her normal, pre-colitis hemoglobin level of around 13-14.

Interviewing her mother, who is the fecal donor, it is suggested that her gut microbiome Faecalibacterium prausnitzii contents be enriched by giving her a specialized diet containing apple pectin, N-acetyl glucosamine, as well as Inulin. Mother and daughter return home to continue in-home fecal enema treatment with the donor taking the Faecalibacterium-enhancement diet.

After 12 weeks of Faecalibacterium-enhanced fecal microbiome treatment, the patient's hemoglobin for the first time rises to between 13.2 and 14.5 and her mother is able to reduce the number of fecal enema treatments from daily to twice weekly.

Three months later the patient has a follow-up colonoscopy. At colonoscopy her mucosa is largely healed and shows the presence of visible, fine mucosal vessels. The luminal stool is in ‘round balls’ indicating from a colonoscopic point of view the healing of severe colitis. There are still occasional areas of blood mixed with stool, indicating that there are spots of mucosa that still need to be healed. Without being bound to any scientific theory, the dramatic change in the patient's hemoglobin indicates that the modification in donor's diet alters the donor stool composition, which in turn modifies the recipient's microbiome composition to help heal her lining in the colon lumen. Again, without being bound to any scientific theory, this ability to reduce the bleeding and oozing due to the diet change saves the patient from having a colectomy—this is mainly due to stimulation of the growth of Faecalibacterium prausnitzii.

1. A pharmaceutical composition comprising a fecal microbiota from a single donor subject administered with one or more Faecalibacterium species or one or more growth stimulating agents for at least one Faecalibacterium species, wherein said fecal microbiota comprises an elevated level of said at least one Faecalibacterium species relative to a control fecal microbiota from the same donor subject not administered with said one or more growth stimulating agents.

2. The pharmaceutical composition of claim Error! Reference source not found., wherein said donor subject ingests said one or more Faecalibacterium species or one or more growth stimulating agents.

3. The pharmaceutical composition of claim Error! Reference source not found., wherein said one or more growth stimulating agents are selected from the group consisting of apple pectin, N-acetyl glucosamine, cysteine, glutathione, riboflavin, and Flavin.

4. The pharmaceutical composition of claim Error! Reference source not found., wherein said elevated level is selected from the group consisting of 1.5-fold or more, 2-fold or more, 2.5-fold or more, 3-fold or more, 3.5-fold or more, 4-fold or more, 5-fold or more, 10-fold or more, 50-fold or more, 100-fold or more, 1000-fold or more, and 10,000-fold or more.

5. A pharmaceutical composition comprising a first plurality of live non-pathogenic microbes capable of inhibiting or antagonizing a Fusobacterium species and a second plurality of live non-pathogenic microbes capable of inhibiting or antagonizing a Mycobacterium species.

6. The pharmaceutical composition of claim 5, wherein said Fusobacterium species is selected from the group consisting of F. necrophorum, F. nucleatum, F. canifelinum, F. gonidiaformans, F. mortiferum, F. naviforme, F. necrogenes, F. russii, F. ulcerans, and F. varium

7. The pharmaceutical composition of any one of claim 5, wherein said Mycobacterium species is Mycobacterium avium, ssp. paratuberculosis (MAP).

8. The pharmaceutical composition of claim 7, wherein said first plurality of live non-pathogenic microbes comprise a Faecalibacterium species

9. The pharmaceutical composition of claim 5, wherein said second plurality of live non-pathogenic microbes comprise one or more, 2 or more, 3 or more, 4 or more, 5 or more, 6 or more, or 7 or more species selected from the anti-myco group consisting of Corynebacterium, Dietzia, Gordonia, Mycobacterium, Nocardia, Segnihparus, Skermania, Tsukamurella, Turicella, Rhodococcus, and Williamsia.

10. The pharmaceutical composition of claim 5, wherein said pharmaceutical composition further comprises a fecal microbe preparation.

11. The pharmaceutical composition of claim 10, wherein said fecal microbe preparation comprises a donor's entire or substantially complete microbiota.

12. The pharmaceutical composition of claim 10, wherein said fecal microbe preparation comprises a non-selected fecal microbiota.

13. The pharmaceutical composition of claim 10, wherein said fecal microbe preparation lacks at least one, at least 2, at least 3, or at least 4 genera of bacteria selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella.

14. A method for treating IBD in a subject in need thereof, said method comprising administering a pharmaceutically active dose of an first antibiotic or probiotic to said subject to inhibit or antagonize a Fusobacterium species.

15. The method of claim 14, wherein said Fusobacterium species is selected from the group consisting of F. nucleatum, F. necrophorum, and F. varium.

16. The method of claim 14, wherein said first probiotic comprises a Faecalibacterium species.

17. The method of claim 14, wherein said first probiotic comprises Faecalibacterium prausnitzii.

18. The method of claim 14, wherein said method further administering a pharmaceutically active dose of an second antibiotic or probiotic to said subject to inhibit or antagonize a Mycobacterium species.

19. The method of claim 18, wherein said Mycobacterium species is Mycobacterium avium, ssp. paratuberculosis (MAP).

20. The method of claim 18, wherein said probiotic comprises one or more, 2 or more, 3 or more, 4 or more, 5 or more, 6 or more, or 7 or more species selected from the anti-myco group consisting of Corynebacterium, Dietzia, Gordonia, Mycobacterium, Nocardia, Segnihparus, Skermania, Tsukamurella, Turicella, Rhodococcus, and Williamsia.
